(19) United States
(12) Patent Application Publication
(10) Pub. No.: US 2018/0360077 A1
(43) Pub. Date: Dec. 20, 2018

(54) PRODUCTION LINE AND METHOD FOR PROCESSING FOOD PRODUCTS

(71) Applicant: Force Technology, Brøndby (DK)

(72) Inventors: Niels KREBS, Hellerup (DK); Keith D. MEEKS, Clovis, CA (US); Aaron ORMOND, Clovis, CA (US)

(21) Appl. No.: 16/063,217

(22) PCT Filed: Dec. 19, 2016
(86) PCT No.: PCT/EP2016/081713

§ 371 Date: Jun. 15, 2018

Related U.S. Application Data
(60) Provisional application No. 62/269,690, filed on Dec. 18, 2015.

(30) Foreign Application Priority Data
Dec. 18, 2015 (DK) PA201570836

(51) Int. Cl. A23L 3/18; A23L 3/30; A23L 3/00; A23L 3/3418; A23L 3/3589

(52) U.S. Cl. CPC A23L 3/185; A23L 3/30; A23L 3/3589; A23L 3/001; A23L 3/3418; A23L 3/003

(57) ABSTRACT

A method for sanitizing food products on a production line (201), and a production line (100) for processing food products (103), comprising: a first processing enclosure (101) and a second processing enclosure (102) and a conveyor system (103) configured to move a food product through the first processing enclosure (101) and onwards through the second processing enclosure (102). The first processing enclosure (101) is coupled to a gas supply system (113) delivering a flow of gas at a gas temperature above 70 degrees Celsius via an orifice (113; 114) to generate a first processing atmosphere within the first processing enclosure (101) exposing at least a portion of the surface of the food products, while travelling through the first processing enclosure, to a first processing temperature (Ts) which is above 60 degrees Celsius. The second processing enclosure (102) is configured with an atomizing nozzle (106; 107) to deliver a spray of a supply of an antimicrobial chemical agent (123) towards the food products (103) travelling through the second processing enclosure.

More particularly there is disclosed a method for processing food products on a production line and a method for sanitizing raw food products on a production line.

### INTRODUCTION/BACKGROUND

It is widely reported that pathogenic contamination of food is a human health hazard in every nation, frequently resulting in human illness, death and economic losses.

Certain highly consumed food products are identified as having a higher than normal risk of harbouring pathogens, such as protein products for example chicken, turkey, pork or beef; and seafood products such as tilapia, swai, salmon or tuna, and produce products for example cantaloupes, broccoli or sprouts; and nuts for example pistachios or almonds and other foods such as cheese or eggs.

Pre-harvest controls are designed as preventative measures to minimize the risk of contamination but pathogens are ubiquitous in the environment and reside in the intestinal track of animals for example chicken, turkey, pork, or cattle which can be transferred during slaughter e.g. by exudate fluid, blood, saliva, mucus, and lymph fluid. Once in the processing facility, microorganisms can detach from the host to cause cross contamination through direct contact with other products, manipulation via mechanics or human workers, cutting equipment, conveyor belts, wash dip tanks, food bins and packaging.

Microbial interventions employed during processing such as steam, high pressure processing, irradiation, ozone, UV, and chemicals are commonly used during food processing to kill microorganisms on food product and food contact surfaces. In many countries food products are bathed and/or sprayed with antimicrobial chemicals to reduce contamination.

Current methods and procedures for many of these methods fail to reduce the microbe burden on food products sufficiently.

As a result, food recalls, food safety alerts and foodborne illness outbreaks are a common occurrence. According to the U.S. Center for Disease Control there are over 48 million cases of food borne illnesses each year in the United States alone, causing 128,000 hospitalizations which claim 3,000 lives each year in the U.S.

Technological improvements for food processing that improve efficacy over existing methods are needed to further reduce the risk of human illness and death, and diminish the economic impact.

### RELATED PRIOR ART

US 2014/322407 discloses a composition comprising hydrogen peroxide as an oxidizing agent and a method for sanitizing a food article comprising the steps of: preparing a use solution by diluting the composition with an aqueous diluent, contacting the use solution with the food article; and allowing sufficient contact time to sanitize the article; and optionally rinsing the article. The composition is applied to the article by spraying or by immersing the article in the solution.

As mentioned in EP 2,478,780 A1 such an antimicrobial composition can be applied with electrostatically accelerated spray.

US 2006/198798-A discloses a peroxygen antimicrobial composition comprising an amine oxide and peroxygen compound. It is claimed that the combination of the two components produces an effective antimicrobial composition, providing a more potent biocide than can be obtained by using these compounds separately. Other components can be added to the composition such as peracetic acid, acetic acid, hydrotrope coupling agents, etc. The composition can be used to sanitize various surfaces such as hard surfaces found in food processing, food service and health care industries.

The peroxygen compound peracetic acid (a.k.a.PAA) is a stronger oxidizer than hydrogen peroxide therefore giving increased efficacy as an antimicrobial.

WO 93/13674-A1 describes a process for exterminating microorganisms residing in a food products in gaseous, liquid and/or pasty form such as milk or milk products. However, the process and the equipment for carrying out the process requires direct contact between a transducer and a the food product, which is not always possible e.g. for food products such as vegetables, nuts, fish and meat, in solid form.

US 2007/059410-A1 relates to cold sterilization and conservation of fruits and vegetables, agricultural and horticultural products, and food stuff with the aid and simultaneous use of different means such as vacuum, ozone, oxygen, carbon-dioxide, argon and UV-C light and ultrasound. However, the embodiments described may require excessive processing time, which is unacceptable connection with commercial food production lines.

Lillard H S: “Decontamination of poultry skin by sonication . . . ” describes that present production practices do not result in Salmonella and Campylobacter free birds and gives an overview of different treatments of food products using ultrasound, sonication, described in the scientific literature. Among other things, it is reported that “There is a paucity of literature on the application of ultrasonics to solid foods such as poultry. It is mentioned that Sams and Feria (1991) exposed pre- and post-chill broiler drumsticks submerged in deionized water to 47 kHz in an ultrasonic cleaning tank. Sonication was for 15 or 30 min at 25 or 40° C. and for shorter intervals (0.5, 2, and 3.5 min) in the presence of lactic acid, with pH adjusted to 2 or 4. However, the embodiments referred to appear to require excessive processing time, which is unacceptable connection with commercial food production lines. Also there appear to be a risk of cross-contamination due to the use of tanks.

US 2004/191374 describes a multi-stage system and method for pasteurizing food products that includes a first processing unit configured to receive the food product and apply an amount of non-thermal energy treatment to the food product which is effective to inactivate one or more key enzymes. A second, subsequent, processing unit is configured to receive the food product from the first processing unit and reduces the population of potentially harmful microorganisms by applying thermal pasteurization. The food product may be tomato paste. Also, the embodiments described therein may require excessive processing time, which is unacceptable connection with commercial food production lines.

DE 39 34 500 A1 describes equipment for sterilizing foodstuff such as spices and dried fruits wherein sterilizing is carried out by both microwaves and ultrasound treatment. The equipment comprises a conveyor belt running through chambers with a microwave generator and a conveyor belt for the foodstuffs. The food conveyor can itself act as an ultrasonic source or can be coupled to a separate source. The equipment comprises a tempering chamber, a moistening chamber, a microwave and ultrasound treatment chamber, and a cooling chamber. A steam inlet is provided for the purpose of adding moist to pepper grains in the microwave and ultrasound treatment chamber. Thus, a wealth of treatments are described, but their efficacy at processing speeds required for commercial food processing lines appear to be below common specifications.

Thus, there is a need for food processing methods applying antimicrobial treatments to food products in solid form with an efficacy useful at processing speeds such as those at commercial food processing lines.

### SUMMARY

It is observed that when a food product is exposed to at least two consecutive antimicrobial treatments comprising a second antimicrobial treatment following a first antimicrobial treatment, wherein the first antimicrobial treatment comprises exposing the food product to heat to elevate its surface temperature, the efficacy of the second antimicrobial treatment is improved over the efficacy of the second antimicrobial treatment when taken alone. There is thus provided:

A method for sanitizing food products on a production line, comprising:
conveying food products through a first processing enclosure and onwards through a second processing enclosure;
generating a first processing atmosphere within the first processing enclosure by supplying a flow of gas, with a gas temperature above 70 degrees Celsius, to the first processing enclosure, wherein at least a portion of the surface of a food product is exposed, while travelling through the first processing enclosure, to a first processing temperature which is above about 60 degrees Celsius; and
inside the second processing enclosure, delivering an antimicrobial treatment to the food products when they travel through the second processing enclosure.

Consequently, a food product is exposed to at least two consecutive antimicrobial treatments, wherein the first antimicrobial treatment comprises exposing the food product to heat to elevate its surface temperature, the efficacy of the second antimicrobial treatment is improved over the efficacy of the second antimicrobial treatment when taken alone.

One reason for this improved efficacy may be that microbes are excessively stressed or stunted by the treatment with heat so that they are more vulnerable when the consecutively following antimicrobial treatment begins. Thus a population of microbes on a portion of the surface of the food product is more effectively killed by the second treatment when following the first treatment.

Thus, the antimicrobial treatment delivered in the second processing enclosure more easily kills those microbes that are stunted and not killed by the processing in the first processing enclosure.

As will be set out in greater below, in some embodiments the second antimicrobial treatment is selected to comprise one or more of: antimicrobial treatment using an antimicrobial agent that is sprayed towards the food product, antimicrobial treatment by rapid chilling of the food product to achieve a surface temperature of at least some portions of a surface of a food product below about 0 degrees Celsius and antimicrobial treatment by exposing the food product to a modified atmosphere.

It should be noted that, the effect of a population of microbes being stunted on a portion of the surface of a food product may remain for a significant period of time after surface temperature of the portion of the food product has reverted to a temperature at or below a temperature of the portion of the food product when it entered the first processing enclosure for heat treatment. This prolonged effect has a synergistic effect with at least the antimicrobial treatment using an antimicrobial agent that is sprayed towards the food product and the antimicrobial treatment by exposing the food product to a modified atmosphere.

The production line can be configured for processing one or more of various types of food products e.g. food products in solid form such as but not limited to: meat e.g. poultry such as chicken, turkey, pheasant, duck, and goose; or beef, veal, pork, lamb, mutton, rabbit, and venison; or seafood such as fish and shellfish; or fruit, berries, vegetables, nuts, and cheeses.

It may be observed that the metabolism of bacteria on said portion of the food product is lowered or arrested during treatment in the first processing enclosure, and wherein the food products are exposed to the treatment in the second processing enclosure before metabolism of bacteria on said portion of the food product has regained a level of metabolism which was measurable when the food product entered the first processing enclosure.

There is an inherent weakness with many food grade antimicrobial agents becoming unstable and degrading before being delivered on the surface of the food product when their temperature deliberately or due to an uncontrolled temperature exposure exceeds room temperature.

However, it appears that at the point in time when an antimicrobial agent becomes unstable an additional or enhanced antimicrobial effect is activated if the microbial organisms are exposed to the antimicrobial agent at that point in time when the antimicrobial agent becomes unstable meaning more reactive.

The Arrhenius equation establishes that chemical reactions occur more rapidly at higher temperatures. The reason for this is the thermal energy relates direction to motion at the molecular level. As the temperature rises, molecules move faster and collide more vigorously, greatly increasing the likelihood of bond cleavages and reformation. The resulting equation is defined as:

k=Ae^{-E}^{ₐ}^{/(RT)}

Wherein k is the rate coefficient, A is a constant, Eₐ is the activation energy, R is the universal gas constant (8.314 J K⁻¹ mol⁻¹), and T is the temperature (in kelvin). At first, k increases exponentially with increasing temperature and then it levels off as it approaches a limit.

This insight is explored in aspects of the method when delivering an antimicrobial treatment to the food products when they travel through the second processing enclosure comprises delivering a spray of an antimicrobial agent towards the food products that travels through the second processing enclosure.

Delivering the antimicrobial agent by spraying it onto the surface of the food product instead of immersing the food product into a tank holding the antimicrobial agent, has at least the advantage that the surface of the food product, which is relatively warm, is not exposed to the cooling impact of the considerable specific heat capacity of the antimicrobial agent in the tank, which is relatively cold to maintain the antimicrobial agent in a stable condition, at least when the antimicrobial agent is an oxidiser. The cooling impact of the antimicrobial agent when sprayed and taking the form of droplets in a mist is much lower than the cooling impact caused by the considerable specific heat capacity of the antimicrobial agent when contained in the tank.

It would generally not be an option, due to stability considerations, to keep the antimicrobial agent in the tank at a temperature about or exceeding the surface temperature of the food product which is deliberately elevated by the processing in the first processing enclosure. Thus, immersion into a tank would detract from the sanitising efficacy obtainable by spraying.

In some embodiments the antimicrobial agent is a food grade antimicrobial chemical agent. In some aspects the antimicrobial agent is selected from the chemical agents allowed for food treatment by the U.S. Code of Federal Regulations or an FDA Food Contract Notification (FCN) or other pertinent regulatory bodies with oversight in the geographical location the method is executed.

As examples:

United States Department of Agriculture Food Safety and Inspection Service provides a list of “safe and suitable ingredients used in the production of meat, poultry, and egg products”. The permitted agents are listed in a directive to food processors—7120.1 rev 29 issued Sep. 9, 2015.

Code of Federal Regulations Title 21 Food and Drugs Chapter 1 food and drug administration department of health and human services subchapter B food for human consumption part 173 secondary direct food additives permitted in food for human consumption subpart D specific usages additives (e.g. part 173.370 Peroxyacids).

Additionally, those items listed with the Inventory of Effective Food Contact Substance Notifications (FCN) available at: http://www.accessdata.fda.gov/scripts/fdcc/?set=fcn

An example is FCN 1389 awarded to Alex C Fergusson LLC (AFCO) that provides regulated concentrations that can be used on meat, poultry, seafood and produce during processing.

Other food grade agents are listed under GRAS notices under U.S. Food and Drug Administration for example GRN No. 435 Preparation consisting of six bacterial monophage specific to Salmonella enterica as an antimicrobial in certain poultry products, fish, shellfish, and fresh and processed fruits and vegetables at 10⁷ plague forming units per gram of food.

(http://www.accessdata.fda.gov/scripts/fdcc/?set=GRASNotices&id=435)

In some aspects the antimicrobial agent is an oxygen-based disinfectant, such as a peroxygen solution.

Typically, antimicrobial chemical agents and especially oxygen-based disinfectants such as a peroxygen compound should be kept at relatively low temperatures to maintain their antimicrobial effect. Therefore, on the one hand, excessive cooling of the food product may occur if dipping or submerging of the food products into a bath of an antimicrobial chemical agent is used as an alternative to spraying with the antimicrobial chemical agent. On the other hand, keeping the antimicrobial chemical agent at a higher temperature and applying dipping or submerging, may degrade shelf life of the food product due to the risk of (thorough) heating and also inhibits ability to maintain constant concentrations of antimicrobial agents delivered in solution.

Consequently, the antimicrobial chemical agent can be dispensed at a sufficiently low temperature to avoid unnecessary degradation of its antimicrobial properties when being an airborne spray while enhancing its antimicrobial efficacy when delivered on the surface of the surface heated food product. This is a great advantage since microbes live on the surface of a food product—and especially since microbes live in small pores, folds, chaps, and slits of the surface which by conventional means can be difficult to reach for effectively reducing a population of microbes.

As mentioned above, an effect is that the antimicrobial effect of the antimicrobial agent meeting a remaining population, after processing in the first processing enclosure, of microbes on the surface of the food product is significantly improved because the likelihood of bond cleavages and reformation of molecules of the antimicrobial agent is increased. This is especially true for an oxygen-based disinfectant.

A variety of peroxygens possesses excellent microbial inhibiting activity under controlled conditions and is sometimes used in chemical sterilization. Hydrogen peroxide (H₂O₂) when put in solution with acetic acid generates a transient molecule named peracetic acid (PAA) that is a high level disinfectant due to the production of highly reactive hydroxyl radical via dissemination of the peroxy bond. The resulting oxidizing agents can denature proteins, disrupt cell wall/membrane permeability and oxidize sulfhydral and sulfur bonds in proteins, enzymes, and other metabolites. They have an additional advantage of producing decomposition products that are nontoxic and biodegradable (water, oxygen, carbon dioxide).

A peroxide is a compound containing an oxygen-oxygen single bond or the peroxide anion, O2-2, The organic peroxides are dominated by the covalent bonds. The oxygen-oxygen chemical bond of peroxide is unstable and easily split into reactive radicals via homolytic cleavage. For this reason, peroxides are found in nature only in small quantities, in water, atmosphere, plants, and animals.

An organic peroxide is defined as R¹—O—O—R², wherein one or both of R1 and R2 contain carbon. The peroxygen bond is the single bond between the two oxygens that can dissociate to create the radicals R1-O and R2-O.

In some aspects the antimicrobial agent comprises an antimicrobial agent of the biological type, such as salmonella bacteriophages. The salmonella bacteriophages may be provided as a slurry of salmonella bacteriophages, e.g. such as Intralytix product marketed under the name “SalmoFresh”.

In some aspects the antimicrobial agent comprises one or more of: peroxygen compound, sodium hypochlorite, chlorine dioxide, hypochlorous acid, hydrogen peroxide, acetic acid, lactic acid, ozone gas in solution, acidified sodium chlorite, potassium hydroxide, sodium hydroxide, citric acid, and a cationic quaternary ammonium compound, such as cetylpridinium chloride.

In some aspects, the surface temperature of the food product is elevated or lowered, while the food product travels through the second processing enclosure, at a rate having a time constant that is relatively short compared to the retention time of the food product in the second processing enclosure. The time constant may be less than 75% of the retention time e.g. less than 50% of the retention time. The time constant may be defined as a 67% temperature level or a 90% temperature level.

In some aspects, delivering an antimicrobial treatment to the food products when they travel through the second processing enclosure comprises a step of performing rapid surface chilling of the food product.

Thereby at least a portion of the surface of the food product is to at least two consecutive treatments, whereby the surface temperature is first rapidly elevated and then immediately after rapidly lowered. Both treatments stress the microbes. Since the second treatment is performed consecutively after the first treatment, microbes are already vulnerable when the second treatment begins. A further stress factor that stunts a population of microbes exposed at least to this dual treatment is the negative and drastic temperature drop from elevated temperatures to low temperatures, say below about 0° C.

One observation is that the population of microbes on the surface will be stunted and brought into a recovery phase since they are heavily stressed in the first processing enclosure; then by applying the growth-inhibiting treatment the microbes remains under stress thus increasing the likelihood that the population will rather be further stunted than recover.

In some aspects one or more of the chilling, cooling and freezing is performed to obtain thoroughly chilling, cooling or freezing, whereby both surface temperature and a core temperature of the food product is lowered.

In some embodiments one or more of rapid chilling, cooling and freezing is performed. The applied rapid chilling, cooling or freezing is applied to lower at least a portion of the surface temperature of a food product to about 0° C. or below 0° C. within about 1-2 minutes or faster.

In some aspects the processing in the first processing enclosure is applied to food products arriving with a surface temperature below 60° C. whereby heat energy is transferred from the gas to the surface of the food product via a gas-to-solid heat transfer transition, where ‘solid’ refers to the surface of the food product, which is not a food product on gaseous, liquid or paste form. The food product may however have a ‘soft’, ‘medium’ or ‘hard’ surface.

The gas is discharged from an outlet, which may be a sound generator as described below, without contact between the outlet and the food product.

By the processing applied in the first processing enclosure, a surface temperature of a food product is elevated from a first surface temperature measured at a point in time when the food product enters the first processing enclosure to a second surface temperature measured at a point in time when the food product leaves the first processing enclosure.

In some embodiments the food product is conveyed to enter the second processing enclosure wherein rapid cooling, chilling or freezing takes place before the surface temperature of at least a portion of the food product falls below the first surface temperature. Thereby a population of microbes on at least a portion of the surface of the food product is exposed firstly to an elevated temperature which increases the likelihood that the population is killed or stunted, and then rapidly thereafter the remaining portion of the population including those stunted is exposed to a low temperature which again, and shortly after the exposure to the elevated temperature, increases the likelihood that the remaining portion of the population is killed. By such a two-phase stress-treatment of the microbes efficacy of the sanitizing is improved over any one of the treatments.

As mentioned above, by firstly exposing a food product to the first processing atmosphere, wherein a first microbe-weakening treatment takes place, and then, secondly, exposing the food product to the second processing atmosphere, wherein a second microbe-weakening treatment takes place, it is observed that a population of microbes on the surface of the food product are more effectively killed than any one of the microbe-weakening treatments.

In some aspects rapid surface chilling is performed by discharging a gas, with a gas temperature below 0° C., inside the second processing enclosure at a sufficient flow rate to cool the surface temperature of at least a portion of the food product to a temperature below about 0° C. within less than about one minute.

In some embodiments, rapid surface chilling is performed by discharging one or more of cold air, an inert gas, liquid Nitrogen, and Carbon Dioxide inside the second processing enclosure as it is known in the art.

In some embodiments, one or both of the flow rate of the gas, the gas temperature, and the time the food product is exposed to the rapid surface chilling treatment is set and/or controlled to obtain a trade-off between obtaining rapid surface chilling and avoiding excessive lowering of a core temperature of the food product.

In some aspects delivering an antimicrobial treatment to the food products when they travel through the second processing enclosure comprises a step of applying a modified atmosphere wherein the volume-percentage of one or both of Nitrogen and Oxygen deviates from 78.08% and 20.95% by more than 1 percentage points.

In some aspects delivering an antimicrobial treatment to the food products when they travel through the second processing enclosure comprises a step of applying a modified atmosphere packaging, MAP.

Thus, when a food product is exposed to at least two antimicrobial treatments, wherein the first antimicrobial treatment comprises exposing the food product to heat to elevate its surface temperature, and wherein the second comprises applying a modified atmosphere, the effect of the treatment by a modified atmosphere is improved over a treatment by a modified atmosphere when taken alone.

As mentioned above, the effect of a population of microbes being stunted on a portion of the surface of a food product may remain for a significant period of time after surface temperature of the portion of the food product has reverted to a temperature at or below a temperature of the portion of the food product when it entered the first processing enclosure for heat treatment. This prolonged effect has a synergistic effect with at least the antimicrobial treatment by exposing the food product to a modified atmosphere.

In some aspects the food products are exposed to the processing atmosphere in the first processing enclosure for a first processing duration; and wherein the first processing duration is in the range of 0.15 to 10 seconds, or in the range of 0.2 to 5 seconds, or less than about 4 seconds.

In some aspects the first processing temperature in the first processing enclosure is in the range of 80 to 95 degrees Celsius. The first processing temperature is measured at the surface of the food product, but at a distance therefrom e.g. at a distance of more than 2 millimetres and less than 5 centimetres.

In some aspects the flow of gas is supplied at a rate of about 15 Kg/hour, 20 Kg/hour, 25 Kg/hour or at a higher rate.

In some aspects a surface temperature of a food product is elevated from a first surface temperature measured at a point in time when the food product enters the first processing enclosure to a second surface temperature measured at a point in time when the food product leaves the first processing enclosure; and wherein the food product is conveyed to enter the second processing enclosure before the surface temperature falls below the first surface temperature.

There are at least two noteworthy effects that are brought about by the above method:

One effect is that a second microbe-weakening treatment of a population of microbes on the surface of the food product is brought into action when a first microbe-weakening treatment of the population of microbes has already significantly stunted the population. Thereby, the first and second microbe-weakening treatments combine synergistic over any one of the treatments.

Another effect is that the antimicrobial effect of the antimicrobial agent meeting a remaining population, after processing in the first processing enclosure, of microbes on the surface of the food product is significantly improved because the likelihood of bond cleavages and reformation of molecules of the antimicrobial agent is increased due to the higher temperature.

In some aspects, the food product is conveyed to enter the second processing enclosure before the surface temperature falls below a threshold level being the first surface temperature plus 10% of the temperature elevation. In some aspects the threshold level is the first surface temperature plus a temperature difference selected from the group of: about 10% of the temperature elevation, about 30% of the temperature elevation, about 50%, about 70% of the temperature elevation, and about 85% of the temperature elevation.

Surface heating of the food products strikes a desired balance between activating the additional or enhanced antimicrobial effect improving efficacy while avoiding the quality degradation or undesired cooking that would follow with thoroughly warming or heating of the food product.

In some aspects the food products are exposed to the processing atmosphere in the first processing enclosure for a first processing duration; and wherein the time it takes a food product from it leaves the first processing enclosure and until it reaches the second processing enclosure is less than about 5 seconds, less than about 2 seconds, or less than about 1 second. However, in general, the time it takes a food product from it leaves the first processing enclosure and until it reaches the second processing enclosure may be longer e.g. up to 20 seconds or 20-40 seconds.

In some aspects the food products are exposed to the first processing atmosphere in the first processing enclosure for a first processing duration, which is sufficiently long to raise the surface temperature of the food product by more than 4 degrees Celsius or more than 10 degrees Celsius.

The processing atmosphere in the first processing enclosure may give the food products a transient exposure to heat that is sufficient to raise the surface temperature of at least a portion of the food products at least 4 degrees Celsius or 10 degrees Celsius.

A raise in surface temperature during treatment in the first processing enclosure may be more than about 10° C., 15° C., 20° C., 30° C., or 40° C. A surface temperature of at least a portion of the food product at the point in time when it leaves the first processing enclosure or immediately thereafter may be up to or even above 50° C., 60° C., or 80° C.

In some aspects the processing atmosphere in the first processing enclosure gives the food products a transient exposure to heat, that is sufficient to raise the surface temperature of at least a portion of the food products at least 4 degrees Celsius or 10 degrees Celsius, while limiting a raise in a core temperature of the food products to a significantly lower temperature than the surface temperature after exposure to the first processing atmosphere.

The speed of the conveyor system, the temperature in the first processing enclosure, and the concentration of the gas in the first processing enclosure are parameters that are set or controlled to achieve a desired surface temperature when the food products leave the first processing enclosure.

In some aspects food products are exposed to the first processing atmosphere in the first processing enclosure for a first processing duration, which is shorter than that required for blanching of the food products at the temperature in the first processing enclosure.

In some aspects the first processing duration, is significantly shorter than that required for blanching of the food products at the temperature in the first processing enclosure.

In some aspects the flow of gas comprises steam supplied to the first processing enclosure at a temperature in the range of about 100 to 140 degrees Celsius or in the range of about 120 to 180 degrees Celsius. The flow of gas, such as steam, is supplied via one or more pipes running from a steam generator to gas outlets, at which the steam is discharged in the first processing enclosure. The temperature of about 100 to 140° C. or about 120 to 180° C. refers to a temperature of the gas, such as steam, in the pipes. In proximity of the outlets, after being discharged, the gas has a somewhat lower temperature of about 100° C. or slightly above or below 100° C.

Herein, steam is water vapour. Steam may comprise water vapour and additives in vaporized form. Vaporized additives in the steam must leave only non-toxic or food grade residues on the food product e.g. when condensed on the surface of the food product.

In some aspects the method comprises a step of applying airborne high intensity and high power acoustic waves to at least a portion of said first processing atmosphere causing it to oscillate substantially at the frequency and substantially with the intensity and power of the acoustic waves.

It has been discovered that the combinational treatment of firstly applying a gas, such as a warm or hot gas, with high intensity sound waves sufficient to disrupt a boundary sub-layer surrounding the food product and then applying an antimicrobial chemical agent, such as peracetic acid, by spraying synergistically activates an improved or additional level of efficacy in combating microorganisms on food products while preserving many desired properties of a food product such as freshness in texture, taste and visual appearance.

The high intensity sound waves are applied in the gas e.g. in connection with discharge of the gas in the first processing enclosure. The high intensity sound waves propagates in the gas, approaching the surface of the food product and disrupting a boundary sub-layer surrounding the food product whereby heat transfer from the gas to the food product takes place faster. In some aspects the processing in the first processing enclosure is applied to food products arriving with a surface temperature below 60° C. whereby heat energy is transferred from the gas to the surface of the food product via a gas-to-solid heat transfer transition, where ‘solid’ refers to the surface of the food product, which is not a food product on gaseous, liquid or paste form. The food product may however have a ‘soft’, ‘medium’ or ‘hard’ surface.

In some aspects said high intensity and high power acoustic waves are ultrasonic acoustic waves. Ultrasonic frequencies may be defined as frequencies in the range about 20 KHz to about 50 KHz.

In some aspects said high intensity and high power acoustic waves are generated by a high intensity and high power acoustic wave generator and has an acoustic sound pressure level at approximately 10 cm from an orifice of said generator selected from the group of:
at least 120 dB,
at least 130 dB,
at least 135 dB,
at least 140 dB,
at least 150 dB,
approximately 130 to approximately 165 dB, and
approximately 130 to approximately 180 dB.

In some aspects high intensity sound or ultrasound is generated by a sound generator of the Hartmann type generator and wherein the pressurized gas is supplied to the sound generator at a pressure in the range of 1.5-5 atm. The sound generator may be a static siren. A static siren generates sound waves without moving parts, at least without moving parts oscillating at the frequencies of the sound generated by the static siren. Thus static sirens, such as the Hartmann generator, may be very robust and continue to work even at long service intervals in production environments. In some aspects the flow of gas is supplied to each sound generator, which may be a static siren, at a rate of about 15 Kg/hour, 20 Kg/hour, 25 Kg/hour or at a higher rate. The temperature of the gas before and after discharge in the first processing enclosure is mentioned above.

Thereby it is possible to achieve a sound pressure level greater than 130 dB, e.g. 132 dB, 134, dB, 136 dB, and up to the highest possible sound pressure achievable, which is approximately 170-180 db. The pressure may be selected to generate a sound pressure in the range of 130-160 dB, above which there is a saturation of the disruptive effect on the sublaminar layer.

In some aspects the food products being sanitized are selected from one of the following groups: poultry, meat, cold seafood, warm seafood, vegetables, fruit, lettuce, berries, nuts, cereal, and cheese.

In some aspects of processing meat e.g. fresh carcasses the processing atmosphere exposes the surface of the carcasses, while travelling through the first processing enclosure, to a raised temperature in the range of 70-100 degrees Celsius, such as to a temperature in the range of 80-95 degrees Celsius. When received at the first processing enclosure, the surface temperature of the fresh carcasses may be in a range of temperatures depending on the type of carcass; cold water seafood can be received at temperatures just above freezing e.g. with a body temperature in the range of 0.5 to 5 degrees Celsius, freshly slaughtered meat can be received at close to body temperature e.g. with a body temperature in the range of 34 to 40 degrees Celsius, and poultry may have a temperature in the range of 30 to 45 degrees Celsius e.g. 32 to 33 degrees Celsius, when received.

Berries have a fragile structure that is easily damaged when exposed to heat. However, berries may carry serious viruses such as Hepatitis and Norovirus. The efficacy of the claimed method and production line makes it possible to treat the berries during only short amounts of time and thus to preserve structure and flavour.

Berries and produce (fruit and vegetables are exposed to a temperature in the range of 70 to 90 degrees Celsius. When received at the first processing enclosure, the surface temperature of the berries may be in the range of 2-25 degrees Celsius or they may be in a frozen condition e.g. with a temperature in the range of −5 to 0 degrees Celsius, or with a lower temperature e.g. below −15 degrees Celsius, such as about −15 degrees Celsius or with a temperature in the range from −5 to 5 degrees Celsius.

For example in the processing of poultry, the temperature of the treatment reaching the poultry product surface in the second processing enclosure is less than 65 degrees Celsius or less than 60 degrees Celsius. The temperature of the spray on the poultry product can ensure that the poultry product is not substantially altered (cooked) by the temperature of the spray.

There is also provided a production line for processing food products, comprising:

a first processing enclosure and a second processing enclosure;
a conveyor system configured to move a food product through the first processing enclosure and onwards through the second processing enclosure;
wherein the first processing enclosure is coupled to a gas supply system delivering a flow of gas at a gas temperature above 70 degrees Celsius via an orifice to generate a first processing atmosphere within the first processing enclosure exposing at least a portion of the surface of the food products, while travelling through the first processing enclosure, to a first processing temperature which is above 60 degrees Celsius;
wherein the second processing enclosure is configured to deliver an antimicrobial treatment to the food products when they travel through the second processing enclosure.

Consequently, since the first processing atmosphere in the first processing enclosure firstly exposes the food product to first microbe-weakening treatment, and then, secondly, the spray of an antimicrobial agent in the second processing enclosure exposes the food product to a second microbe-weakening treatment, it is observed that a population of microbes on the surface of the food product is more effectively killed than any one of the microbe-weakening treatments.

In some aspects one or both of the first processing enclosure and the second processing enclosure is a cabinet or chamber. The cabinets or chambers may be arranged in contact with one another such that the exit way of the first processing enclosure abuts with an entry way of the second processing enclosure. Thereby exposure to external or uncontrolled atmospheres can be minimized.

In some aspects the orifice delivering a flow of gas is comprised by a multitude of orifices coupled to the gas supply system delivering a flow of gas at a raised temperature. Thereby an improved distribution of the processing atmosphere exposing the surface of the food products to a raised temperature is achievable.

In some aspects the second processing enclosure is configured with an atomizing nozzle to deliver a spray of a supply of an antimicrobial chemical agent towards the food products travelling through the second processing enclosure.

In some aspects the antimicrobial agent is an oxygen-based disinfectant, such as a peroxygen solution.

In some aspects the second processing enclosure is configured to perform rapid surface chilling of the food product.

In some aspects rapid surface chilling is performed by discharging a gas, with a gas temperature below 0° C., inside the second processing enclosure at a sufficient flow rate to cool the surface temperature of at least a portion of the food product to a temperature below about 0° C. within less than about one minute.

In some aspects the second processing enclosure (102) is configured to apply a modified atmosphere wherein the volume-percentage of one or both of Nitrogen and Oxygen deviates from 78.08% and 20.95% by more than 1 percentage points.

In some aspects the second processing enclosure is configured to apply a modified atmosphere packaging, MAP.

In some aspects the production line is configured to expose the food products to the processing atmosphere in the first processing enclosure for a first processing duration; and wherein the first processing duration is in the range of 0.15 to 10 seconds, or in the range of 0.2 to 5 seconds, or less than about 4 seconds.

In some aspects the first processing temperature in the first processing enclosure is in the range of 80 to 95 degrees Celsius.

In some aspects the flow of gas comprises steam supplied to the first processing enclosure (201) at a temperature in the range of about 100 to 140 degrees Celsius or in the range of about 120 to 180 degrees Celsius

In some aspects the first processing enclosure is configured to apply airborne high intensity and high power acoustic waves to at least a portion of said first processing atmosphere causing it to oscillate substantially at the frequency and substantially with the intensity and power of the acoustic waves.

In some aspects said high intensity and high power acoustic waves are ultrasonic acoustic waves.

In some aspects said high intensity and high power acoustic waves are generated by a high intensity and high power acoustic wave generator and has an acoustic sound pressure level at approximately 10 cm from an orifice of said generator (100) selected from the group of:
at least 120 dB,
at least 130 dB,
at least 135 dB,
at least 140 dB,
at least 150 dB,
approximately 130 to approximately 165 dB, and
approximately 130 to approximately 180 dB.

In some aspects high intensity sound or ultrasound is generated by a sound generator of the Hartmann type generator and wherein the pressurized gas is supplied to the sound generator at a pressure in the range of 1.5-5 atm.

In some aspects the production line comprises a first storage tank for storing the antimicrobial agent, a second storage tank containing an antimicrobial agent solution, and a compressor for pressurizing the second storage tank and driving the antimicrobial agent solution towards one or more of the nozzles.

The antimicrobial agent is driven through the one or more nozzles via one or more of a piping system or tubing system or hose system connecting the second tank and the nozzles.

In some aspects the atomizing nozzle is configured to deliver an air assisted induction charged electrostatic spray (AAIC-ES).

In some aspects the production comprises a steam generator delivering pressurized steam to the sound generator. The steam generator may deliver a steam of water to the sound generator which emits the sound waves with discharge of the steam through which the high intensity sound waves propagates towards the surface of the food products at which a sub-laminar layer of surrounding air is disrupted. Thereby the heat transfer to the surface of the food product is accelerated. This allows for fast processing times and less heat damage to the inner structure of the food product. The residue from this treatment is mainly water which can be drained from the first processing enclosure via a drainage.

In some aspects the production comprises a wall separating a first processing volume enclosed by the first processing enclosure and a second processing volume enclosed by the second processing enclosure; wherein the wall has an opening forming a passage through which the conveyor and a food product conveyed thereon can pass.

The wall serves the purpose of restricting inflow of gas from the first processing enclosure to the second processing enclosure. However, since at least the food products have to pass from the first processing enclosure to the second processing enclosures, a limited inflow will take place.

The wall may be multi-layered wall, such as a double layered wall or it may be single-layer wall. The wall may be thermally insulated by a thermally insulating layer.

### BRIEF DESCRIPTION OF THE FIGURES

A more detailed description follows below with reference to the drawing, in which:

FIG. 1 shows a portion of a production line for processing food products;

FIG. 2 shows a first production line for processing food products; and

FIG. 3 shows surface temperature, T, of a food product as a function of distance, x, along a production line;

FIGS. 4a, 4b and 4c show surface temperature, T, of a food product as a function of distance, x, along a production line comprising a first and a second processing enclosure;

FIG. 5 shows a portion of a production line for processing food products, wherein the first and second processing enclosure are abutting one another;

FIG. 6 shows an exemplary cooling facility;

FIG. 7 shows an exemplary chilling facility;

FIG. 8 shows a portion of a production line for processing food products comprising a chilling facility located downstream of the heating facility;

FIG. 9 shows a portion of a production line for processing food products comprising a modified atmosphere facility;

FIG. 10 shows a portion of a production line for processing food products comprising a chilling facility and an antimicrobial agent spraying facility;

FIG. 11 shows the first processing enclosure with a temperature controller; and

FIG. 12 shows the second processing enclosure with a temperature controller.

### DETAILED DESCRIPTION

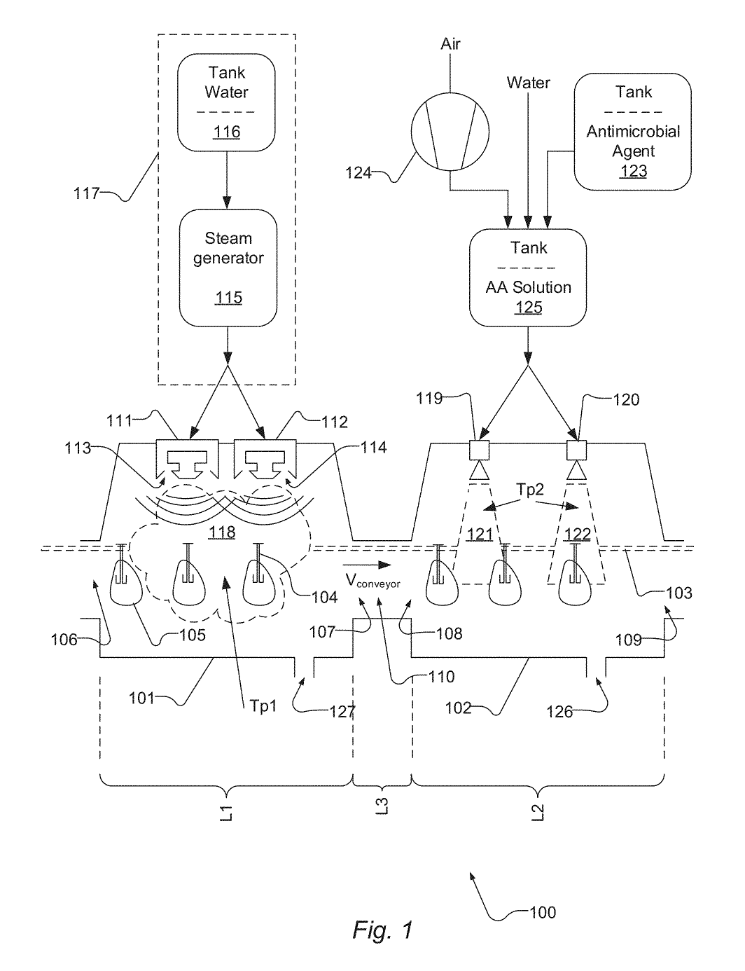

FIG. 1 shows a portion of a production line for processing food products. The portion of the production line is generally designated by reference numeral 100 and comprises a first processing enclosure 101 and a second processing enclosure 102. Food products are generally designated 105 and are shown hanging on hooks 104 suspended from a conveyor 103. However, in some embodiments the conveyor may be configured differently e.g. as a belt conveyor whereon the food products are conveyed; the belt of the conveyor or sections thereof may comprise a mesh whereon the food products are carried. The mesh may be e.g. 70% open mesh which provides access to the surface of the food product and even that portion of the surface of the food product which faces the conveyor belt.

The conveyor 103 moves the food products 105 in a direction which is generally denoted a down-stream direction and at a velocity V_{conveyor}. The conveyor 103 is loaded with food products 105 at a location upstream of first processing enclosure 101 and unloaded at a location downstream of the second processing enclosure 102. The conveyor 103 follows a path that enters the first processing enclosure 101 via an entry way 106 and leaves it via an exit way 107; and then enters the second processing enclosure 102 via an entry way 108 and leaves it via an exit way 109. The exit way 107 and the entry way 108 may be connected by a duct 110 or take the form of an opening in a common, single- or multilayer, wall that separates the interior volume of first processing enclosure 101 from the interior volume of the second processing enclosure 102. The aperture formed by one or more of the exit way 107, the entry way 108 and the duct 110 is, on the one hand, sufficiently large that the conveyor and the food products it carries can pass with a sufficiently large clearance to avoid collision during normal operation of the production line. On the other hand, the size of aperture is limited to limit inflow of gas from the first processing enclosure 101 to the second processing enclosure. The aperture may have a cross-sectional area which is less than a cross-sectional area of the first processing enclosure e.g. less than 75% or less than 50% or less than 25% of the cross-sectional area of the first processing enclosure.

The path that the conveyor 103 follows has a first section which extends inside the first processing enclosure, a second section which extends inside the second processing enclosure and a third section which connects the first and second section.

In some embodiments the first section of the conveyor extends linearly or substantially linearly through the first processing enclosure. Likewise, in some embodiments the second section of the conveyor extends linearly through the second processing enclosure. The first section, the second section and the third section of the conveyor has lengths L1, L2 and L3, respectively. The length of the first section may be from about 10 cm up to about 4 meters, e.g. about 1.5 meters. The length of the second section may be in the range of 1-3 meters, e.g. about 1.5 meters.

The length of the third section of the conveyor, extending between the first and the second processing enclosure, may be less than 50 cm, less than 30 cm, less than 15 cm or less than about 10 cm; it may be up to 2 meters, e.g. up to 1 meter. In some aspects, the food product is conveyed to enter the second processing enclosure (102) before the surface temperature of a portion of a food product falls below a threshold level being a first surface temperature plus 10% of the temperature elevation; wherein the first surface temperature is the surface temperature at the portion of the food product when it entered the first processing enclosure or immediately before. In some aspects the threshold level is the first surface temperature plus a temperature difference selected from the group of: about 10% of the temperature elevation, about 30% of the temperature elevation, about 50%, about 70% of the temperature elevation, and about 85% of the temperature elevation.

The sound generators 111 and 112 may be arranged in a pattern e.g. along one or more lines that follows the first section of the path of the conveyor.

The nozzles 119 and 120 may be arranged in a pattern e.g. along one or more lines that follows the second section of the path of the conveyor.

In some embodiments the first processing enclosure comprises one sound generator; in other embodiments the first processing enclosure comprises a multitude of sound generators.

In some embodiments the second processing enclosure comprises one nozzle; in other embodiments the second processing enclosure comprises a multitude of nozzles.

The first processing enclosure 101 is equipped with one or more sound generators 111 and 112 with a respective orifice 113 and 114 through which gas is delivered to the interior of the first processing enclosure 101. The gas is supplied to the sound generators 111 and 112 via pipes from a steam generator 115. The steam generator 115 receives water from a tank 116 and is configured to deliver a steam at a pressure of 1.5 to 5 bar to each sound generator 111, 112. The tank 116 and the steam generator 115 are commonly referred to by reference numeral 117.

Thereby a first processing atmosphere 118 is generated within the first processing enclosure 101. The first processing atmosphere 118 represents the temperature, sound pressure, and composition of matter (e.g. steam) that the food products are exposed to when travelling through the first processing enclosure.

One or more of the sound generators 111 and 112 are configured as a static siren e.g. as a Hartmann generator to generate high intensity and high power acoustic waves to at least a portion of said first processing atmosphere causing it to oscillate substantially at the frequency and substantially with the intensity and power of the acoustic waves.

In some embodiments the sound generators 111 and 112 are arranged below the food products when they travel on the conveyor. They may also be arranged to emit sound from the sides of the first enclosure 101 or from above the food products—or arranged in a configuration comprising any combination thereof. There may be installed one sound generator or a multitude of sound generators.

The second processing enclosure 102 is equipped with one or more atomizing nozzles 119 and 120 that deliver a spray of an antimicrobial agent towards at least a portion of the food products 105 that travels on the conveyor 103 through the second processing enclosure 102. The spray is illustrated by the cones 121 and 122. The spray may be emitted at a substantially constant rate throughout normal operation of the processing line or it may be gradually adapted to the traffic of food products on the conveyor or other conditions.

The antimicrobial agent is stored in a first tank 123 to provide a local supply of the antimicrobial agent. The supply of antimicrobial agent is mixed with water in a second tank 125 to provide a solution of the antimicrobial agent. The water may be potable ground water delivered at tap ambient temperatures, anywhere from 1-30° C. but most typically in the range of 10-23° C. The tank 125 is pressurized by compressed air from a compressor 124. Via an outlet of the tank 125 the solution of the antimicrobial agent is supplied under pressure to the atomizing nozzles 119 and 120.

The mixture proportion of antimicrobial agent to water is chosen to deliver a predefined of concentration of the antimicrobial agent as it is known in the art to the nozzles 119 and 120.

In some embodiments the nozzles 119 and 120 or at least one of them is configured as an atomizing nozzle. In some aspects, the atomizing nozzle is configured to deliver an air assisted induction charged electrostatic spray (AAIC-ES).

In some embodiments, a flowrate controller is installed to secure that the antimicrobial chemical agent is supplied to each atomizing nozzle at a flowrate in the range of 80-200 millilitres per minute.

The tank 123 storing the supply of the antimicrobial agent is located to avoid excessive heating e.g. by direct sunlight and at a temperature below 30° C. (86 F). For instance if an antimicrobial agent such as peroxygen, or another oxidizer is exposed to excessive heating there would be a risk of disrupting equilibrium of the agent which in turn would increase reactivity and potentially result in an exothermic reaction which could result in a fire hazard. A peroxygen compound may comprise a peroctanoic acid or a peracetic acid.

The temperature, designated Tp2, of the antimicrobial agent in the second processing enclosure 102 is about the same as the temperature of the water supplied and mixed with the antimicrobial agent as mentioned above.

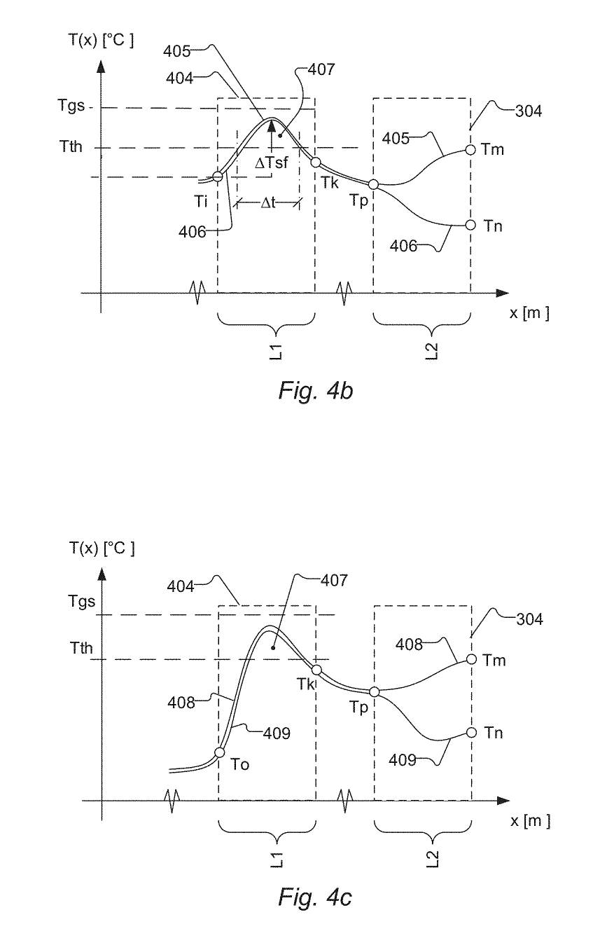

In some aspects the temperature of the antimicrobial agent in the second processing enclosure 102 is controlled by an automatic controller to obtain a desired surface temperature at the exit way of the second processing enclosure or a desired temperature profile for the surface temperature of the food product during the course of the processing applied in the second processing enclosure. The desired surface temperature at the exit way of the second processing enclosure may be higher, lower or substantially the same as the surface temperature of the food product at a point in time when the food product entered the second processing enclosure—this is illustrated in connection with FIGS. 4a, 4b and 4c. The automatic controller may e.g. control the temperature by controlling the flow of two sources of water at different temperatures, by a heater or a cooler or a combination thereof. In some aspects the automatic controller controls temperature via a source of gas such as a source of air. In some aspects the gas is fully or partly re-circulated in the second processing enclosure under control of the automatic controller so as to maintain a desired temperature as measured by a temperature sensor in the second processing enclosure and/or to obtain a desired surface temperature of the food products as measured automatically or at intervals by an operator by a contact less temperature sensor such as an IR camera.

In some embodiments residues produced by the processing carried out in the first processing enclosure 101 are drained from the enclosure via drainage 127; and the residues produced by the processing carried out in the second processing enclosure 102 are drained from the enclosure via drainage 126. For instance when the gas supplied in the first processing enclosure is steam, water is drained from drainage 127 and when the antimicrobial agent is an oxidizer such as a peroxygen, water is drained from drainage 126. Peracetic acid decomposes into acetate which eventually decomposes into carbon dioxide and water in a series of reactions occurring over time. The immediate fluid collected from drainage of the spray would most likely still have at least some acetic acid, acetate, possibly some PAA still decomposing, possibly some hydrogen peroxide still oxidizing etc.

It should be noted that the conveyor 103 may comprise a single conveyor line, a system of like conveyors, or a system of different types of conveyors. In some embodiments the conveyor comprises a station which hands over food products from one conveyor to another. In some embodiments the conveyor comprises a conveyor of a type conveying the food products in a suspended manner. In some embodiments the conveyor comprises a conveyor of a type conveying the food products on a belt, e.g. a belt with a mesh structure or sections e.g. comprising a mesh which is 50% open, e.g. 70-80% open.

In some embodiments the conveyor is configured to one or more of: inclining, turning, flipping upside/down, or rotating a food product to expose different portions of the food product and different portion of the surface thereof to treatment while travelling through one or both of the first and second processing enclosure. Thereby, e.g. the atomizing nozzles 119 and 120 may be located to deliver the spray from above and/or from the sides of the food product, while being able to reach lower portions of the food product as well.

In some embodiments the atomizing nozzles 119 and 120 are arranged below the food products when they travel on the conveyor.

In some embodiments the conveyor is configured to drop off food products or hand-over food products or branch off the production line such that different processing paths may be provided for production purposes or for quality inspection purposes.

In some embodiments one or both of the first processing enclosure and the second processing enclosure are configured with a longitudinal and upwardly facing slot in a top portion, such as a top plate or roof, of the one or more processing enclosures. Thereby, the conveyor 103, e.g. a rail thereof, can extend above one or both of the processing enclosures and be arranged such that suspension devices (such as a shackle and/or hook), extending from the conveyor and carrying the food products run in the longitudinal and upwardly facing slot, while the food products are conveyed below the top portion of the processing enclosures and inside the processing enclosures. The slot is configured to allow the suspension devices to run therein with a sufficient clearance, but with a narrow slot such that the processing atmosphere inside the one or more processing enclosures is substantially confined by the processing enclosure. The slot may be closed by a gasket that is normally closed, but opens, e.g. due to flexibility, at locations at which a suspension device passes.

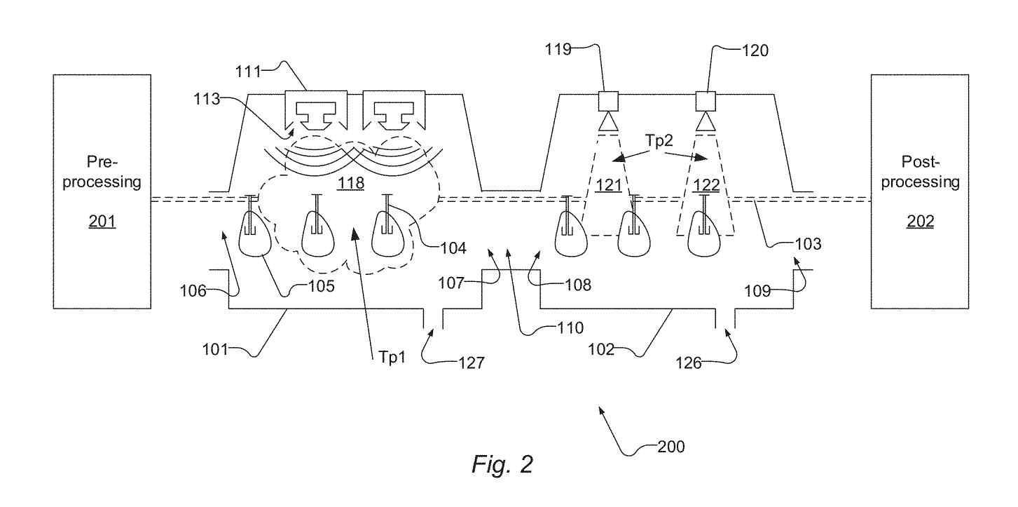

FIG. 2 shows a first production line for processing carcasses. The first production line generally designated by reference numeral 200 can be configured for processing one or more particular types of food product among different types of food products.

The production line 200 comprises the portion of a production line 100 described in greater detail in connection with FIG. 1, a pre-processing facility 201, which may comprise a single pre-processing station or a pre-processing production line, and a post-processing facility 202 which may comprise a single post-processing station or a post-processing production line.

In some embodiments the pre-processing facility 201 comprises a station for lowering the core temperature of the food product. The core temperature of a food product may be lowered by circulating cold air or another type of gas, e.g. comprising CO2, around the food products for a sufficient amount of time. In some aspects the food products are subjected to a cold or super-cold atmosphere in a so-called freeze tunnel. The food product may thereby reach a chilled of frozen state. The core temperature of a food product may alternatively be lowered by submerging the food product into a bath containing a liquid with a sufficiently cold temperature to reach a desired core temperature of the food product within an acceptable retention time in the bath.

In some embodiments the post-processing facility 202 comprises a station for lowering the core temperature of the food product. The lowering of the core temperature may be performed as described above.

It should be noted that the pre-processing facility 201 and the post-processing facility 202 refer to any section of the production line upstream of the first processing enclosure and downstream of the second processing enclosure, respectively.

The pre-processing facility 201, the portion of a production line 100, and the post-processing facility 202 are configured or adapted to the processing of the one or more particular types of food product such that a desired or acceptable food quality is achieved.

In some embodiments the processing atmosphere in the first processing enclosure 101 gives the food products a transient exposure to heat, that is sufficient to raise the surface temperature of at least a portion of the food products at least 4 degrees Celsius or 10 degrees Celsius, while limiting a raise in a core temperature of the food products to a significantly lower temperature than the surface temperature after exposure to the first processing atmosphere.

In some embodiments the food products are exposed to the first processing atmosphere in the first processing enclosure 201 for a first processing duration, which is shorter than that required for blanching of the food products at the temperature in the first processing enclosure.

Thus, since one or both of heat capacity and specific heat capacity may vary significantly among different types of food products and sizes of cut-out portions, and since food products may have complex shapes, it is a complex task to quantify the first processing duration, the temperature of the first processing atmosphere, and the entropy of the first processing atmosphere. In this respect the following will enable a person skilled in the art to configure a production line as described above.

Processing of different types of food products is described below in accordance with different embodiments. It should be noted that unless parameters and processing steps are indicating otherwise, the parameters and processing steps of one embodiment may be applied in another embodiment.

In general the embodiments comprise a processing step of applying an antimicrobial agent such as an oxygen such as peroxygen solution, e.g. peracetic acid (PAA) or peroctanoic acid. However, this step may be interchanged by a step of rapid chilling or rapid cooling or a step of modified atmosphere processing such as modified atmosphere packaging as described further below.

### First Embodiment (Processing of Poultry, Whole Birds)

Examples of food products processed in accordance with the first embodiment may comprise whole birds of chicken, turkey, ostrich, game hen, squab, guinea fowl, pheasant, duck, goose, emu, or a combination thereof.

At the pre-processing facility, livestock of birds are received, stunned and then slaughtered to deliver eviscerated bird carcasses. The eviscerated bird carcasses have a temperature of about 32-45° C. After evisceration the bird carcasses are washed in an inside-outside bird washer which uses a large amount of water and thereby typically lowers the temperature to below about 32° C. e.g. about 28° C. The temperature depends among other things on the temperature of the water used in the washer which may change with the time of year.

At a temperature in the range of about 25-34° C. the eviscerated and washed bird carcasses are conveyed to the first processing enclosure 101, wherein the surface temperature of the carcasses or at least a portion thereof is raised to a temperature which is more than about 4° C. higher than the surface temperature of the carcasses or at least a portion at the time when they entered the first processing enclosure 101. A raise in surface temperature during treatment in the first processing enclosure may be more than about 10° C., 15° C., 20° C., 30° C., or 40° C. A surface temperature of at least a portion of the food product at the point in time when it leaves the first processing enclosure may be up to or even above 50° C., 60° C., or 80° C.

The temperature in the first processing enclosure is e.g. in the range of 80-95° C. The duration of the first processing is e.g. in the range of 1-4 seconds.

The eviscerated bird carcasses are immediately thereafter conveyed to the second processing enclosure 102, wherein the antimicrobial agent is applied. When the antimicrobial agent is a peroxygen solution, e.g. peracetic acid (PAA) or peroctanoic acid, the concentration thereof is selected to be in the range of 1-2000 ppm. Alternative, food grade antimicrobial agents may be e.g. Nisin, Trisodium Phosphate, or cetylpyridinium chloride.

The eviscerated bird carcasses are thereafter conveyed to the post-processing facility 202 which may comprise a cooling station wherein a step of cooling of the sanitized carcasses is performed. Cooling may be performed by lowering the carcasses while travelling on the conveyor into a tank containing chilled water with a temperature of 1-4° C. for up to about 90 minutes, e.g. 50-60 minutes. Subsequently, and as a step of the post-processing, the sanitized and cooled carcass may be packaged e.g. by steps comprising wrap packaging.

In some embodiments, in an alternative to lowering the food product into a tank, cooling is performed using an air chiller and exposing the food product to air chilling for about 80-100 minutes, e.g. for about 90 minutes.

### Second Embodiment (Poultry, Cut-Up Parts)

Poultry carcasses may be processed as described in connection with the first embodiment above, with the change that the carcasses proceed to a cut-up station after cooling, optionally via storage at a temperature of 0-4° C. for e.g. about 24 hours.

For processing of cut-up parts of poultry, pre-processing steps may involve air-chilling wherein the cut-up parts of poultry are exposed to a chilled circulating air, e.g. at a temperature of 0.5 to 4° C.

At a temperature in this range the cut-up are conveyed to the first processing enclosure 101, wherein the surface temperature of the cut-up parts or at least a portion thereof is raised to a temperature which is more than 4° C. higher than the surface temperature at the time when the part entered the first processing enclosure. A raise in surface temperature during treatment in the first processing enclosure may be more than about 10° C., 15° C., 20° C., 30° C., or 40° C. A surface temperature of at least a portion of the food product at the point in time when it leaves the first processing enclosure may be up to or even above 50° C., 60° C., or 80° C.

The temperature in the first processing enclosure is e.g. in the range of 80-95° C. The duration of the first processing is e.g. in the range of 0.5-5 seconds.

The cut-up parts are immediately thereafter conveyed to the second processing enclosure 102, wherein the antimicrobial agent is applied as disclosed in connection with the first embodiment.

The cut-up parts are thereafter conveyed to the post-processing facility 202 which may comprise a step of chilling as described above. Subsequently, and as a step of the post-processing, the sanitized cut-up parts may be packaged e.g. by steps comprising wrap packaging; e.g. by modified atmosphere packaging.

### Third Embodiment (Meat, Pork/Beef)

Examples of food products processed in accordance with the third embodiment may comprise cut-up parts of pork or beef.

At the pre-processing facility 201, livestock are received, stunned and then slaughtered, comprising bleeding, head/shank removal, skinning, evisceration, splitting, and trim to deliver carcasses. The carcasses then have a temperature of typically less than 40° C.

At a temperature in the range below 40° C. the parts are conveyed to the first processing enclosure 101, wherein the surface temperature of the parts or at least a portion thereof is raised to a temperature which is more than 4° C. higher than when the parts entered the first processing enclosure. A raise in surface temperature during treatment in the first processing enclosure may be more than about 10° C., 15° C., 20° C., 30° C., or 40° C. A surface temperature of at least a portion of the food product at the point in time when it leaves the first processing enclosure may be up to or even above 50° C., 60° C., or 80° C.

The temperature in the first processing enclosure is e.g. in the range of 80-95° C. The duration of the first processing is e.g. in the range of less than 5 seconds, e.g. less than 4 or 3 seconds, e.g. less than 1 second, e.g. about 0.5 seconds.

The carcasses are immediately thereafter conveyed to the second processing enclosure 102, wherein the antimicrobial agent is applied as disclosed in connection with the first embodiment.

The cut-up parts are thereafter conveyed to the post-processing facility 202 which may comprise a step of chilling as described above. Subsequently, and as a step of the post-processing, the sanitized cut-up parts may be packaged e.g. by steps comprising wrap packaging; e.g. by modified atmosphere packaging. Optional further steps comprise cold storage or freezing.

The concentration of the antimicrobial agent, being e.g. PAA, is selected to be in the range of 1-4000 ppm, e.g. 230-2000 ppm, e.g. up to 4000 ppm.

In some aspects a 1% Lactic acid solution is used instead of PAA and the concentration is then about 10,000 ppm Lactic acid.

### Fourth Embodiment (Cold Seafood)

At the pre-processing facility 201, raw seafood material is received in a holding tank, and then subjected to one or more of bleeding, rinse, filleting, skinning, boning, trimming and grading.

The temperature of the seafood material when arriving at the first processing enclosure, after being processed at the pre-processing facility 201, is in the range of 0-8° C.

At a temperature in the range below about 8° C. the seafood parts are conveyed to the first processing enclosure 101, wherein the surface temperature of the parts or at least a portion thereof is raised to a temperature which is more than 4° C. higher than when the parts entered the first processing enclosure. A raise in surface temperature during treatment in the first processing enclosure may be more than about 10° C., 15° C., 20° C., 30° C., or 40° C.

The seafood material passes through the second chamber where it is exposed to the aforementioned spray of an antimicrobial agent and then proceeds to packaging and either freezing or chilling

The post-processing facility may comprise one or more of Individual Vacuum Packaging (IVP), Individual Quick Freeze (IQF) and air-chilling.

The seafood material may be individually vacuum packed (IVP) and then subjected to individual quick freeze (IQF) at temperatures of about −20° C. to thoroughly freeze the product.

Alternatively, the fish fillet may be IQF and then IVP or it may be subjected to a glaze such as a Sodium Phosphate solution or 2% salt solution, then place in freezer room to freeze product. Another potential form of product involves either placing the fillet in an IVP or merely wrapping in a plastic film and holding the product in chill rooms where product temperature is between 0-4° C.

The concentration of the antimicrobial agent, being e.g. PAA, is selected to be in the range of 1-1000 ppm e.g. 50-230 ppm.

### In a Fifth Embodiment (Warm Seafood)

Warm seafood may be processed in a similar way as cold seafood, except that the temperature of the raw seafood material when arriving at the first processing enclosure is in the range of about 8-30° C.

The surface temperature of at least some of the raw seafood material when arriving at the first processing enclosure is below about 30° C. At a temperature in the range below about 30° C. the seafood parts are conveyed to the first processing enclosure 101, wherein the surface temperature of the parts or at least a portion thereof is raised to a temperature which is more than 4° C. higher than when the parts entered the first processing enclosure. A raise in surface temperature during treatment in the first processing enclosure may be more than about 10° C., 15° C., 20° C., 30° C., or 40° C.

The concentration of the antimicrobial agent is selected to be in the range of 1-1000 ppm e.g. 50-230 ppm.

### Sixth Embodiment (Vegetables)

The temperature of vegetables when arriving at the first processing enclosure is in the ranges of about 7-29; 10-32; and 7-30° C. for e.g. broccoli, peppers, and sprouts, respectively.

At a temperature in the range below about 30° C. the vegetables are conveyed to the first processing enclosure 101, wherein the surface temperature of the parts or at least a portion thereof is raised to a temperature which is more than 4° C. higher than when the pieces entered the first processing enclosure. A raise in surface temperature during treatment in the first processing enclosure may be more than about 10° C., 15° C., 20° C., 30° C., or 40° C.

Steps performed at the pre-processing facility 201 may comprise: harvest inspection to assess quality, weighing, removal of leaves, hot-water-plunge blanching followed by cold water-plunge.

Steps performed at the post-processing facility 202 may comprise one or both of air chilling and freezing.

The concentration of the antimicrobial agent is selected to be in the range of 1-500 ppm e.g. 10-80 ppm

### Seventh Embodiment (Fruit)

Processing of fruit may comprise processing of fruit such apples, pears, kiwi and different types of leguminous fruit. When arriving at the first processing enclosure, fruit may have a temperature in the range of e.g. 7-29° C. or e.g. 10-32° C.

Pre-processing in the pre-processing facility 201 may comprise, harvest-inspection, grading and rinsing to remove dirt.

At a temperature in the range of e.g. 7-29° C. or e.g. 10-32° C. the pieces of fruit are conveyed to the first processing enclosure 101, wherein the surface temperature of the parts or at least a portion thereof is raised to a temperature which is more than 4° C. higher than when the pieces entered the first processing enclosure. A raise in surface temperature during treatment in the first processing enclosure may be more than about 10° C., 15° C., 20° C., 30° C., or 40° C.

Post-processing in the post-processing facility 202 may comprise one or more of 1) cooling of whole fruit items and 2) cut-up into pieces of fruit, followed by packaging and chilling and optionally cold storage.

The concentration of the antimicrobial agent, being PAA, is selected to be in the range of 1-500 ppm e.g. below 80 ppm.

### Eighth Embodiment (Berries)

Processing of berries may comprise processing of strawberries, blueberries, cherries and other types of berries. When arriving at the first processing enclosure, berries may have a temperature in the range of e.g. 15-25° C. However, berries may also be processed in a frozen condition in which case the temperature of the berries is below 0° C.

The berries are conveyed to the first processing enclosure 101, wherein the surface temperature of the parts or at least a portion thereof is raised to a temperature which is more than 4° C. higher than when the pieces entered the first processing enclosure. A raise in surface temperature during treatment in the first processing enclosure may be more than about 10° C., 15° C., 20° C., 30° C., or 40° C.

Pre-processing in the pre-processing facility 201 may comprise, harvest-inspection, grading and washing to remove dirt.

Post-processing in the post-processing facility 202 may comprise packaging and optionally freezing or chilled storage.

The concentration of the antimicrobial agent is selected to be in the range of 1-500 ppm e.g. below 80 ppm.

### Ninth Embodiment (Nuts, Cereal)

Processing may comprise processing of one or both of nuts, almonds and cereal. When arriving at the first processing enclosure, nuts or cereal may have a temperature in the range of e.g. 15-25° C.

At a temperature in the range of e.g. 15-25° C. the pieces are conveyed to the first processing enclosure 101, wherein the surface temperature of the parts or at least a portion thereof is raised to a temperature which is more than 4° C. higher than when the pieces entered the first processing enclosure. A raise in surface temperature during treatment in the first processing enclosure may be more than about 10° C., 15° C., 20° C., 30° C., or 40° C. A surface temperature of at least a portion of the food product at the point in time when it leaves the first processing enclosure may be up to or even above 50° C., 60° C., or 80° C.

Pre-processing in the pre-processing facility 201 may comprise, receiving of raw material, removal of orchard debris, drying, processing by shear rollers to remove hull, hull aspiration, processing by shear rollers to remove shell, shell aspiration and grading by gravity separators or classifier screens.

Post-processing in the post-processing facility 202 may comprise packaging and optionally freezing or chilled storage.

The concentration of the antimicrobial agent is selected to be in the range of 1-500 ppm e.g. below 80 ppm.

With respect to the above embodiments and in general, it should be noted that, it is very difficult to quantify an exact range of temperatures before and after the different process steps, because of all the differences in production processes. Also the temperature change on the food item is dependent on many variables such as the mass of the food item, the heat coefficient, and specific heat of the food item, the ambient room temperature, the intensity of the steam, the duration of steam exposure, etc.

However, a person skilled in the art will understand that the processing taking place in the first and second processing enclosures can be adjusted—without undue experimentation—to achieve effective sanitizing of the food items while achieving a visual attractive appearance of the food item and maintaining innate colour and texture qualities.

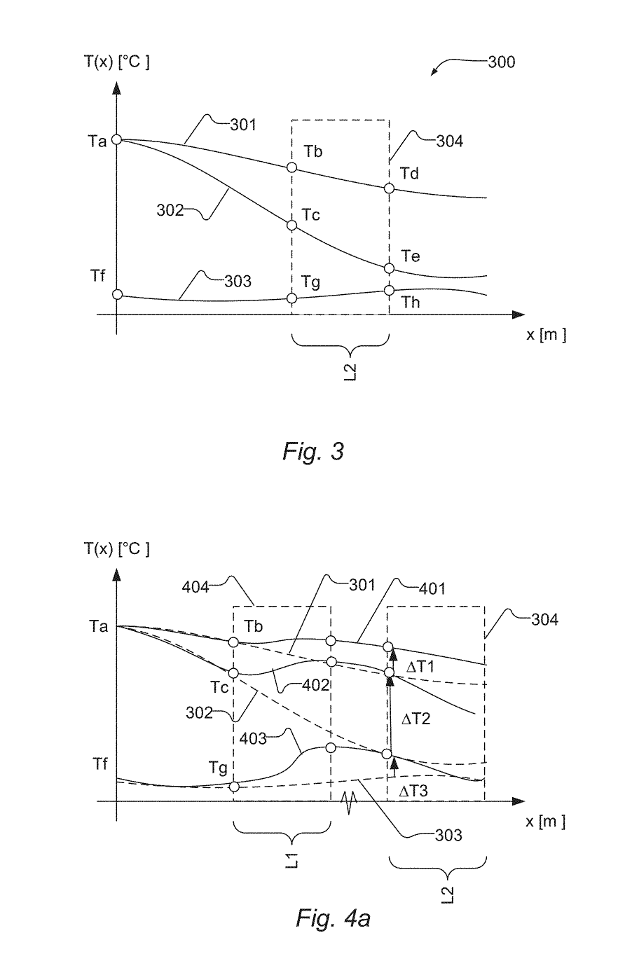

FIG. 3 shows temperature, T, of the surface of a food product as a function of distance, x, along a production line. The coordinate system 300 has an x-axis indicating the distance x [m] from a point upstream of the second processing enclosure 102 and to a point downstream of the second processing enclosure 102. In this figure, the second processing enclosure is shown as a dashed line box 304 and the path of the conveyor through the second processing enclosure has a length L2. It should be understood that the temperature of the surface of food product typically is not completely uniform—variations across the surface of the food product occur. Thus, the temperature, T, of the surface of a food product refers to a predetermined portion of the food product. For consistency, the temperature is illustrated for the same portion of the food product. The surface temperature of the food product or a surface temperature of a portion of a food product may be measured by a non-contact thermometer, sometimes denoted an IR camera, or a temperature gun, or in another way. A non-contact thermometer may give a temperature reading by measuring, at a distance, infrared radiation from one or more points or areas on the food product.

A collection of curves 301, 302 and 303 illustrate examples of how the surface temperature of a food product may develop along the production line—assuming that the first processing enclosure is NOT in operation. The y-axis of the coordinate system indicates the temperature [° C.].

The curves 301 and 302 start at the upstream point at a relatively high temperature Ta and then drops relatively slowly and quickly towards temperatures Tb and Tc, respectively, at the point of entry to the second processing enclosure 304. At the point of exit from the second processing enclosure 304 the temperature of a food product has dropped further to temperatures Td and Te, respectively.

The curve 303 starts at the upstream point at a relatively low surface temperature Tf and then remains roughly at the same temperature level until the food product reaches the entry way of the second processing enclosure 304 at temperature Tg. Due to the relatively low temperature Tg, the temperature of a relatively cold food product may increase while being processed in the second processing enclosure 304 to a temperature Th.

FIG. 4a shows temperature, T, of the surface of a food product as a function of distance, x, along a production line comprising a first and a second processing enclosure. The collection of curves 301, 302, and 303 shown in FIG. 3 are repeated in this figure as dashed lines for comparison with a collection of curves 401, 402 and 403. In this figure, the first processing enclosure is shown as a dashed line box 404 and the path of the conveyor through the first processing enclosure has a length L1.

The collection of curves 401, 402 and 403 illustrate examples of how the surface temperature of a food product may develop along the production line—assuming that the first processing enclosure is in operation.

As can be seen by comparing curve 301 and curve 401, the first processing enclosure raises the surface temperature of a food product by a temperature increase ΔT1. In a similar way, by comparing curve 302; 303 and curve 402; 403, respectively, the first processing enclosure raises the surface temperature of a food product by a temperature increase ΔT2 and ΔT3, respectively.

Thus, it is illustrated that the first processing enclosure 101 raises the surface temperature of food product such that the surface temperature of a food product when it leaves the first processing enclosure 101 is elevated relative to when it entered the first processing enclosure. The surface temperature of a food product leaving the first processing enclosure need not be higher than surface temperature of the food product when it entered the production line, but it may be elevated to a higher temperature than when it entered the production line.

It may happen on a production line, upstream of the first processing enclosure 101, that the surface temperature of a food product drops due to the relatively low room temperature experienced upstream of the first processing enclosure or due to chilling occurring in connection with e.g. washing of the food product. This is illustrated by curves 301; 401 and 302; 403 and takes place typically when the temperature Ta of the food product at the start of the production line is relatively high; e.g. for freshly slaughtered carcasses.

It may also happen that the surface temperature is roughly unchanged or even elevated due to processing such as washing upstream of the first processing enclosure. This is illustrated by curves 303 and 403 and takes place typically when the temperature Tf of the food product at the start of the production line is relatively low; e.g. for seafood.

It should be understood that the temperature of the surface of food product or a portion thereof may be different from a core temperature of the food product. The core temperature may be measured at a core location such as at a geometrical centre of the food product or at a significant depth into the food product. The core temperature may be measured by inserting a temperature probe into food product. Thus, there may be a temperature gradient between the core location and the surface.

Since the food product is conveyed at a speed through the processing enclosures, the surface temperature may change more volatilely, than the core temperature, when exposed to a treatment in one or both of the first processing enclosure and the second processing enclosure. The core temperature of the food product may remain substantially unchanged or at least more stable than the surface temperature. The steepness of the temperature gradient will depend on, among other things, the heat capacity of the food product or a portion of the food product, the thickness of the food product where the core temperature is measured, and the core temperature of the food product at the time when it enters the first processing enclosure.

FIG. 4b also shows the temperature, T, of the surface of a food product as a function of distance, x, along a production line. The food product has a surface temperature, Ti, when the food passes the entry way to the first processing enclosure. The first processing atmosphere within the first processing enclosure generated by the supply of a flow of gas, with a gas temperature, Tgs, raises the surface temperature of the food product as shown by the curves 405 and 406. Within the first processing enclosure, or a region thereof, the surface temperature of the food product reaches and exceeds a temperature level, Tth, during a period of time, Δt. The period of time, Δt, is depicted along the x-axis since the food product is conveyed at a velocity, such as a substantially constant velocity, through the first processing enclosure. The food product has a retention time within the first processing enclosure which depends on the extend, L1, of the first processing enclosure and the velocity of the food product being conveyed through the enclosure. The retention time may be in the range of 0.1-10 seconds e.g. 0.5-5 seconds. The period of time, Δt, during which the surface temperature is above the temperature level, Tth, may be in the range of 0.1-5 seconds or longer or shorter.

In some aspects the heat applied to the food product, by the gas temperature being higher than the surface temperature, Ti, at the time when the food passes the entry way to the first processing enclosure, causes the surface temperature of the food product to raise and transiently reach a temperature peak at or above 72° C. when passing through a section of the first processing enclosure. At the point in time when the food product leaves the first processing enclosure, passing the exit way, the surface temperature may have dropped below 72° C. or it may stay at or above 72° C. when the food product leaves the first processing enclosure. Meanwhile, while the food product travels through the first processing enclosure, a core temperature of the food product remains within a core temperature range with an upper limit significantly lower than 72° C. In some aspects the following parameters:
temperature of the gas,
the flow of the gas (volume), and
the retention time of the food product within the first processing enclosure
   are selected for a predefined food product or range of food products with comparable characteristics such that the surface temperature of the food product raises, by an increased surface temperature, ΔTsf, from an entry temperature, Ti, lower than 72° C. and transiently reach a temperature peak at or above 72° C., while a core temperature of the food product raises less than a desired fraction of 30%, such as less than 20% or less than 10%, of the increased surface temperature.

The gas temperature of the gas supplied to the first processing enclosure is set sufficient high that the discharged gas has a temperature higher than 72° C., such as above 80° C. or above 90° C. in proximity of the gas outlet, after being discharged. The gas is supplied at a sufficient flow volume that the surface temperature transiently reaches the surface temperature of 72° C. or above. The retention time of the food product (the period of time it stays in the first processing enclosure) is selected sufficiently short that the core temperature of the food product raises less than the desired fraction of the increased surface temperature.

Thus, if a food product enters the first processing enclosure at a surface temperature and a core temperature about 40° C., the surface temperature may peak at e.g. 78° C., which is an increased surface temperature, ΔTsf, of 40° C. The core temperature is then kept below 40° C.+0.3 times 38° C.=51.4° C., wherein the 0.3 is the desired fraction mentioned above.

It should be noted that the surface temperature may peak within a centre region of the first processing enclosure or in a region at the exit way of the first processing enclosure.

As also shown in FIG. 4b, by the curve 405, the surface temperature may increase during the treatment applied in the second processing enclosure, compared to the surface temperature, Tk, at the exit way of the first processing enclosure to surface temperature, Tm, at the exit way of the second processing enclosure.

Also, as shown by the curve 406, the surface temperature may decrease to surface temperature, Tn, during the treatment applied in the second processing enclosure. During the course of the treatment applied in the second processing enclosure the core temperature of the food product may change to a larger degree than during the treatment applied in the first processing enclosure, especially so when the treatment applied in the second processing enclosure is in the form of a mist or bath.

In some aspects the core temperature is not raised above about 50° C. e.g. not above 45° C.

It should be noted that the surface temperature, Tk, of the food product at the exit way of the first processing enclosure is higher than the surface temperature, Ti or To, at the entry way of the first processing enclosure. This is illustrated in connection with FIG. 4a. Thus, the first processing enclosure elevates the surface temperature of the food product. In combination therewith, and as illustrated in connection with FIGS. 4b and 4c, a transient and peak surface temperature of the food product may be reached at a point while the food product travels through the first processing enclosure or at the exit way.

FIG. 4c also shows the temperature, T, of the surface of a food product as a function of distance, x, along a production line. The food product has a surface temperature, To, when the food passes the entry way to the first processing enclosure. By means of the curves 408 and 409 it is illustrated that the surface temperature, To, when the food passes the entry way to the first processing enclosure is lower than the surface temperature Ti.

The lower surface temperature, To, may represent cut pieces, such as fillets, of meat or bird whereas the higher surface temperature, Ti, may represent the surface temperature of vegetables, fruit, or fresh carcasses

The core temperature of the food product may be substantially at the same temperature as the surface temperature or within a range thereof. This may be the case for e.g. seafood. Correspondingly, the surface temperature may increase more steeply.

Reference numeral 407 refers to the area below one of the curves 405; 406; 408; 409 representing the surface temperature and above the temperature level, Tth. The area may be quantified using a temperature-times-time unit [° C.·Sec.]. The area may be in the range of less than 140 [° C.·Sec.], e.g. less than 80 [° C.·Sec.].

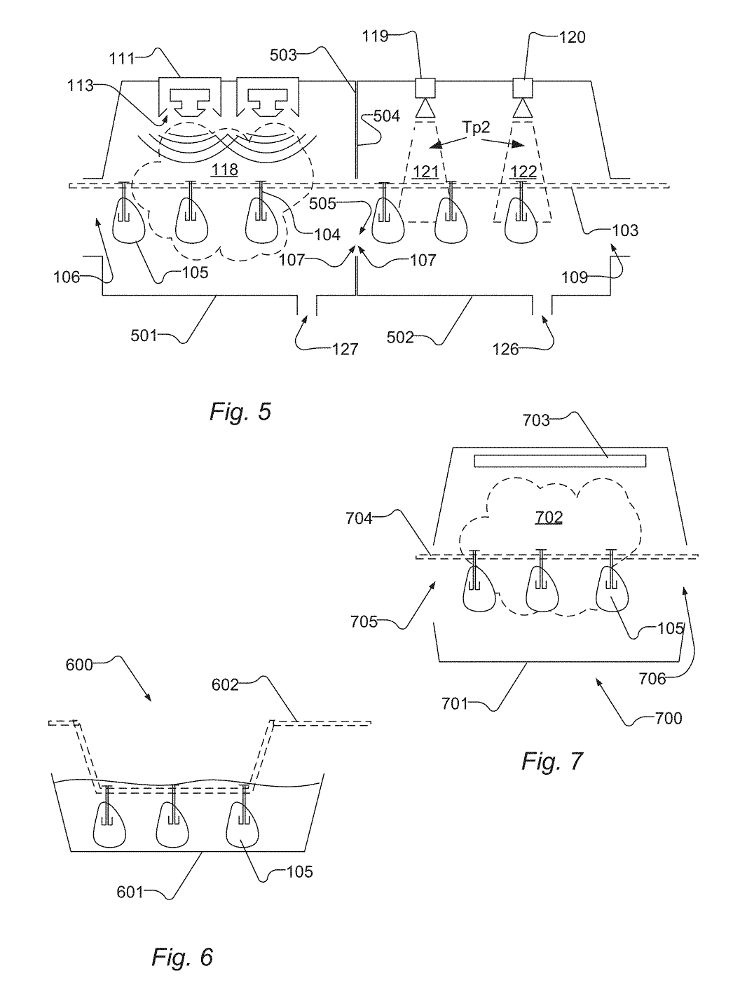

FIG. 5 shows a portion of a production line for processing food products, wherein the first and second processing enclosure a abutting one another. Thereby, travel time of food products being conveyed from the first processing enclosure to the second processing enclosure may be made very short.

Here, the first processing enclosure and the second processing enclosure are referred to by reference numerals 501 and 502. The processing enclosures 501 and 502 have abutting walls 503 and 504, respectively. On the contrary, the processing enclosures 101 and 202 have slightly inclined walls.

The abutting walls may abut as closely as practically possible e.g. below a few centimetres or the processing enclosures may be installed or configured to have a deliberate space of e.g. 10 to 50 centimetres e.g. with air between them.

In an alternative embodiment the first processing enclosure 501 and the second processing enclosure 502 are configured as semi-detached processing enclosures e.g. by sharing a wall separating the interior of the first processing enclosure from the interior of the second processing enclosure. However, the wall has an opening to allow for passage of the conveyor and food products carried thereon.

FIG. 6 shows an exemplary cooling facility. The cooling facility generally designated 600 comprises in this example a tank 601 containing chilled water with a temperature of 1-4° C. The food products 105 are lowered into the tank 601 by a conveyor section 602 that brings about an immersion of the food products into the chilled water contained in the tank. The immersion may be brought about by a track of the conveyer being downwardly deflected over at least a portion of the tank. The conveyor thereby moves the food product 105 through the chilled water in the tank.

In some embodiments the chilled water in the tank may be another liquid medium than water or a solution comprising an agent such as an antimicrobial agent.

The size of the tank 601, the length of the conveyor section 602 and the speed of the conveyor are configured to give the food product a sufficient immersion time to give the food product a desired temperature. The immersion time is e.g. less than 100 minutes, e.g. 90 minutes.

FIG. 7 shows an exemplary chilling facility. The chilling facility generally designated 700 comprises a processing enclosure 701 with an entry way 705 and an exit way 706 enabling a conveyor section 704 to convey the food products 105 into, through and out of the processing enclosure 701.

The processing enclosure 701 is configured with a cooling member 703 which cools and circulates a gas such as air inside the processing enclosure thereby creating an atmosphere 702 which chills the food products 105. The conveyor section is connected or coupled to the conveyor 103.

One or both of the cooling facility 600 and the chilling facility 700 are configured to apply a sufficient amount of cooling energy to rapidly cool and chill, respectively, the food products to a sufficient low temperature to inhibit growth of microbes or to increase the likelihood that a population of microbes is killed or destroyed or stunted.

In some embodiment one or both of the cooling facility 600 and the chilling facility 700 are located upstream of the first processing enclosure 101 or downstream of the second processing enclosure 102. In some embodiments one or both of the cooling facility 600 and the chilling facility 700 are located downstream of the first processing enclosure 101 and upstream of the second processing enclosure 102—thus being situated between the first and second processing enclosures.

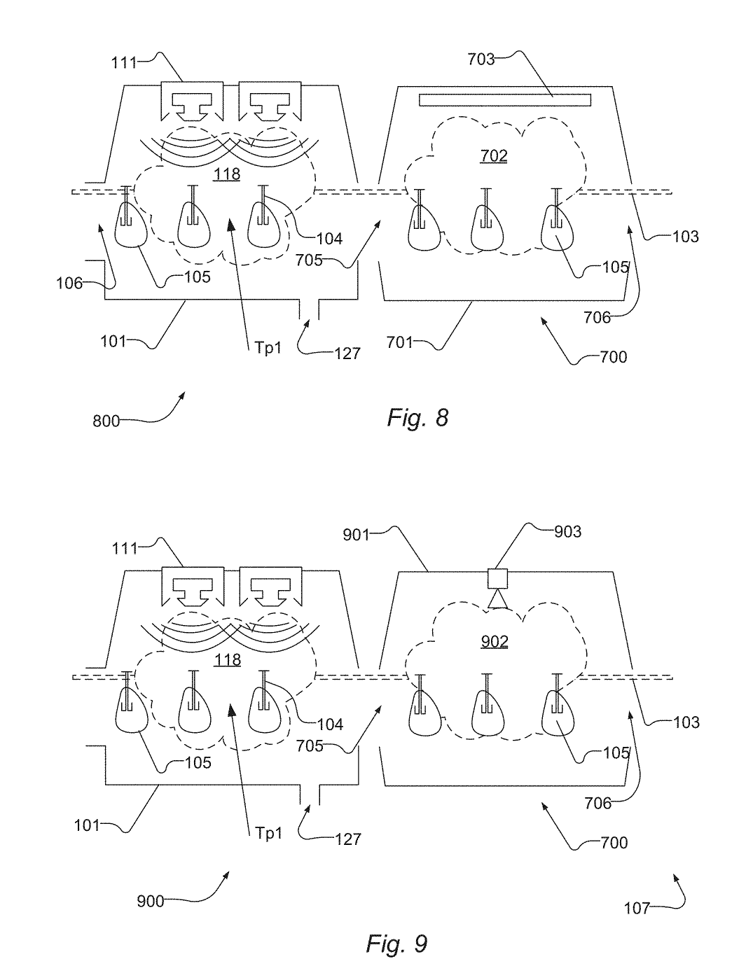

FIG. 8 shows a portion of a production line for processing food products comprising a chilling facility located downstream of the heating facility. The portion of the production line is designated by reference numeral 800 and comprises the above-mentioned first processing enclosure 101 and the above-mentioned processing enclosure 701. The conveyor 103, which may be a system of conveyors, is arranged to extend through the first processing enclosure and onwards through the processing enclosure 701. Other details shown are described above.

FIG. 9 shows a portion of a production line for processing food products comprising a modified atmosphere facility. The portion of the production line is designated by reference numeral 900 and comprises the above-mentioned first processing enclosure 101 and a further processing enclosure 901. Inside the further processing enclosure 901 an antimicrobial treatment is delivered to the food products 105. The antimicrobial treatment comprises generating a modified atmosphere 902 inside the further processing enclosure 901. The modified atmosphere 902 has a volume-percentage of one or both of Nitrogen and Oxygen that deviates from 78.08% and 20.95% by more than 1 percentage points, such as by more than 2, 5, or 8 percentage points or more.

A supply of gas (not shown) generating the modified atmosphere 902 is delivered via a nozzle 903. The gas may be mixed from different supplies of gas as it is known in the art.

In some embodiments a so-called modified atmosphere packaging, MAP, step is performed inside the processing enclosure 901. Then a packaging machine (not shown) is installed inside the processing enclosure 901 and the conveyor 103 comprises a section conveying packaged food products out of the processing enclosure.

In some embodiments there is installed a station or processing enclosure lowering the core temperature of a food product, and thus also the surface temperature, in readiness for modified atmosphere packaging, MAP. The first processing enclosure 101 is located for processing the food product, and processes the food product as described above, following the station or processing enclosure which lowers the core temperature of a food product and before the processing enclosure 901, wherein a step of modified atmosphere packaging, MAP, is performed. On the one hand, the core temperature is lowered e.g. by submerging the food product into a bath or by circulating cold gas around the food product as described above. On the other hand, while travelling through at least a section of the first processing enclosure 101, a surface temperature of the food product is raised, by an increased surface temperature, to transiently reach a temperature at or above 72° C., while the core temperature of the food product raises less than 30% of the increased surface temperature.

Thereby, by the processing step of lowering the core temperature of the food product, the food product can reach or keep a core temperature that is appropriate for packaging by the MAP step, while antimicrobial heat-treatment is applied in the first processing enclosure immediately before the food product is subjected to packaging. Thereby, a total bacterial count and bacterial growth are effectively controlled before and up to the point of packaging. The MAP step may be a final processing step in the manufacture of a packaged food product.

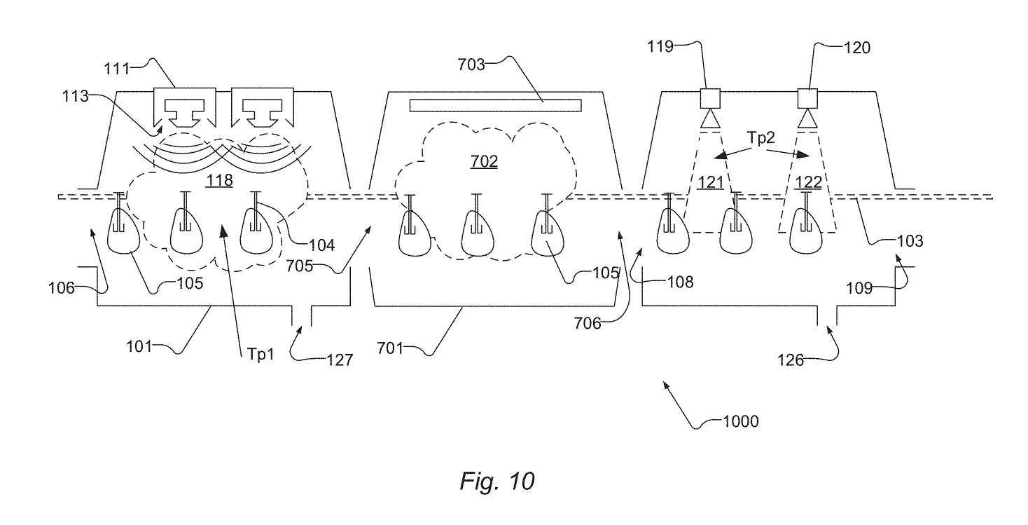

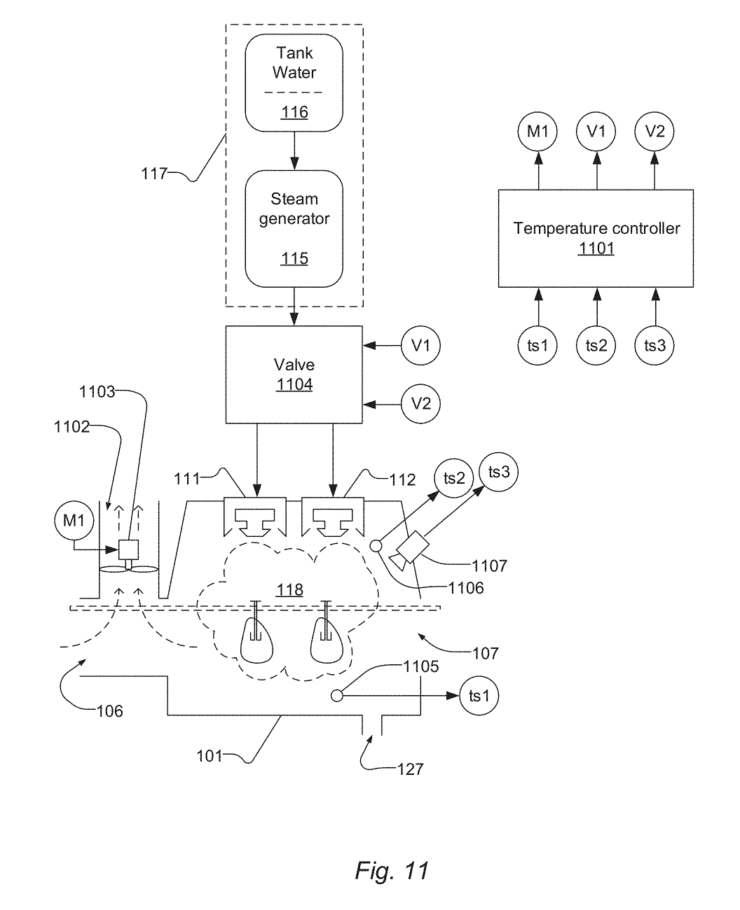

FIG. 10 shows a portion of a production line for processing food products comprising a chilling facility and an antimicrobial agent spraying facility. The portion of the production line is designated 800 and the elements thereof are described above. It should be noted that the chilling facility 700 may be replaced by the cooling facility 600.

FIG. 11 shows the first processing enclosure comprising a temperature controller. The temperature controller 1101 is coupled by a wired or wireless connection to one or more sensors and one or more actuators as illustrated by correspondingly labelled circles.

Temperature controller 1101 receives input from the sensors as indicated by the circles labelled ts1, ts2 and ts3. The temperature controller 1101 generates output to the actuators as indicated by the circles labelled M1, V1 and V2.

The temperature controller 1101 receives input from one or more sensors, which may comprise a temperature sensor 1105 and a temperature sensor 1106. The temperature sensors 1105 and 1106 may be of the thermocouple type, or of another type suitable for measuring the (gas) temperature of the processing atmosphere inside the first processing enclosure at one or more points therein.

The temperature controller 1101 may additionally or alternatively receive input from a remote sensing temperature sensor 1107, such as an IR camera, configured and arranged to measure surface temperature of the food products at an area thereof. Temperature measurements output from the remote sensing temperature sensor 1107 may be electronically gated to filter out temperature measurements not measured at the surface of a food product. The gating may use a sensor being activated by a suspension device of the conveyor or by thresholding temperature measurements.

Thus, one or more of the temperature of the processing atmosphere, at one or more points, and the surface temperature of the food products may be input to the temperature controller. The temperature controller may be a PID type, a PI type or of another linear or non-linear type. The controller may be of the on/off type outputting binary, of/off control signals or it may output control signals with a finer quantization.

The temperature controller 1101 controls a fan 1103, which is installed in a duct 1102 and configured to evacuate some of the processing atmosphere 118 continuously or periodically at regular or irregular intervals. The fan 1103 may comprise one or more motorized fans.

The duct 1102 is shown as an upwardly, vertical, straight duct, but it may comprise a horizontal portion or extend along a substantially horizontal course. The duct 1102 is coupled to a path through which the food products are conveyed into the first processing enclosure. In other embodiments the duct 1102 is installed from a top portion or roof of the first processing enclosure. In some embodiments the duct is arranged at the exit way of, or at an exit path from, the first processing enclosure. In some embodiments there is installed a duct both at the entry way, as shown, and at the exit way. The duct at the exit way may be configured similar to the duct 1102 at the entry way or different therefrom. Similarly, a fan is installed in the duct at the exit way and the fan is configured to evacuate some of the processing atmosphere 118 continuously or periodically at regular or irregular intervals under control of the temperature controller 1101. The fan 1103 and the fan in the duct at the exit way may be activated simultaneously in the same way or at different times. The fan 1103 is comprised by a class of exhaust devices.

In some embodiments the temperature controller 1101 controls the controllable valve 1104 which regulates the supply of gas to the sound generators 111 and 112 or to other types of gas outlets. The supply of gas may be regulated in an on/off way, controlling gas supply to individual sound generators or outlets or group-wise. The controllable valve 1104 may comprise multiple valves coupled in a manifold configuration with multiple individually or group-wise controllable outputs to respective outlets or group of outlets.

In some embodiments the temperature controller 1101 controls a valve regulating the pressure of the steam supplied to the controllable valve 1104 to regulate one or both of the volume and pressure of steam supplied. The pressure may be regulated within a predefined range of pressures such that the sound generators continue to operate as desired.

Item 1: There is provided a method for sanitizing food products on a production line (200), comprising:
conveying food products (105) through a first processing enclosure (201);
generating a first processing atmosphere within the first processing enclosure (201) by supplying a flow of gas, with a gas temperature above 80 degrees Celsius, to the first processing enclosure (201), wherein at least a portion of the surface of a food product is exposed, while travelling through the first processing enclosure (101), to a first processing temperature (T1) which is above about 72 degrees Celsius;
controlling one or both of a temperature of the first processing atmosphere and a surface temperature of a food product travelling through the first processing enclosure (101), comprising:
   automatically measuring one or both of a gas temperature inside the first processing enclosure and a surface temperature of a food product travelling through the first processing enclosure;
   using a temperature controller (1101) to control one or both of a gas temperature inside the first processing enclosure and a surface temperature of a food product travelling through the first processing enclosure by one or both of:
      regulating an exhaust device (1103) coupled to the first processing enclosure (101) to evacuate therefrom some of the processing atmosphere (118) or to forgo evacuating some of the processing atmosphere (118); and
      regulating the flow of gas;
   in response to temperature measurements performed by the automatic measuring.

Item 2: In some embodiments according to item 1, the surface temperature of the food product is measure by a remote sensing temperature sensor.

Item 3: In some embodiments according to item 2, the surface temperature of the food product is controlled to reach or exceed a predetermined first temperature level, such as a temperature level of 72 degrees Celsius.

Item 4: In some embodiments according to any of the items above, one or both of a gas temperature inside the first processing enclosure and a surface temperature of a food product travelling through the first processing are controlled, to prevent one or more of measured temperatures from exceeding a predetermined second temperature level, such as a temperature level of 85 degrees Celsius.

Item 5: In some embodiments according to any of the items above, the flow of gas is regulated by regulating the pressure of the flow of gas within a predefined range, such as within a range of 1.5 to 5 bar to each of a set of gas outlets.

Item 6: In some embodiments according to any of the items above, the flow of gas is regulated by one or more valves supplying gas to one or a group of gas outlets; wherein one or more of the valves are on/off (open/closed) controlled.

For the sake of completeness, encircled label ‘M1’ designates a connection between the temperature controller 1101 and the fan 1103; encircled label ‘V1’ designates a connection between the temperature controller 1101 and the steam generator 115; encircled label ‘V2’ designates a connection between the temperature controller 1101 and the valve 1104. The temperature controller 1104 is coupled to the sensors in a similar way.

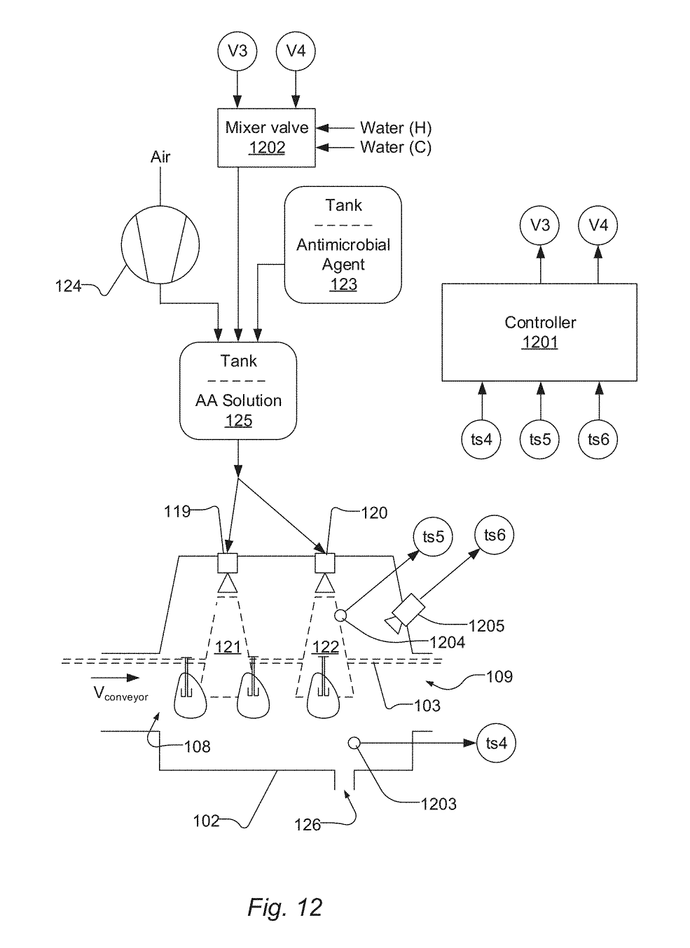

FIG. 12 shows the second processing enclosure comprising a temperature controller. The temperature controller 1201 is coupled by a wired or wireless connection to one or more sensors and one or more actuators as illustrated by correspondingly labelled circles.

Temperature controller 1201 receives input from the sensors as indicated by the circles labelled ts4, ts5 and ts6. The temperature controller 1201 generates output to the actuators as indicated by the circles labelled V3 and V4.

The temperature controller 1201 receives input from one or more sensors, which may comprise a temperature sensor 1203 and a temperature sensor 1204. The temperature sensors 1203 and 1204 may be of the thermocouple type, or of another type suitable for measuring the temperature of the processing atmosphere, which may be a mist, inside the second processing enclosure 102 at one or more points therein.

The temperature controller 1201 may additionally or alternatively receive input from a remote sensing temperature sensor 12025, such as an IR camera, configured and arranged to measure surface temperature of the food products at an area thereof. Temperature measurements output from the remote sensing temperature sensor 1205 may be electronically gated to filter out temperature measurements not measured at the surface of a food product. The gating may use a sensor being activated by a suspension device of the conveyor or by thresholding temperature measurements.

Thus, one or more of the temperature of the processing atmosphere, at one or more points, and the surface temperature of the food products may be input to the temperature controller. The temperature controller may be a PID type, a PI type or of another linear or non-linear type. The temperature controller may be of the on/off type outputting binary, of/off control signals or it may output control signals with a finer quantization.

The temperature controller 1201 controls a mixer valve 1202 which receives water supplies from a first source, Water (H) at a relatively warm temperature and from a second source, Water (C), at a relatively low temperature. The mixer valve 1202 may comprise a first valve regulating inlet from the first source in response to a control signal, V3, and a second valve regulating inlet from the second source in response to a control signal, V4. The first valve and the second valve maybe arranged in a manifold configuration with an outlet at which the water has a temperature causing the surface temperature of the food products or the mist in the cones 121 and 122 or at another location in the second processing enclosure to have a desired temperature.

In some embodiments one or both of the remote sensing temperature sensors 1107 and 1205 aim at a food product from a direction at which the food products approach the remote sensing temperature sensor 1107 as the food products are conveyed on the conveyor. The remote sensing temperature sensor 1107 may be located above, below or to the sides, but out of the way of the food products' path on the conveyer.

Item 7: There is provided a method for sanitizing food products on a production line (200), comprising:
conveying food products (105) through a second processing enclosure (102);
generating a supply of an antimicrobial agent solution to a spray nozzle;
inside the second processing enclosure (102), delivering an antimicrobial treatment to the food products (103), by spraying, via the spray nozzle, the antimicrobial agent solution towards the food products, at least at a point in time when they travel through the second processing enclosure (202);
controlling one or both of a temperature inside the second processing enclosure (202) and a surface temperature of a food product travelling through the first processing enclosure (101), comprising:
   automatically measuring one or both of a temperature inside the second processing enclosure (102) and a surface temperature of a food product travelling through the second processing enclosure;
   using a temperature controller (1201) to control one or both of a temperature inside the second processing enclosure (102) and a surface temperature of a food product travelling through the second processing enclosure by regulating the temperature of the supply of the antimicrobial agent solution to the spray nozzle in response to temperature measurements by the automatically measuring.

Item 8: In some embodiments according to item 7, the surface temperature of the food product is measure by a remote sensing temperature sensor.

Item 9: In some embodiments according to item 8, one or both of a temperature inside the second processing enclosure (102) and a surface temperature of a food product travelling through the second processing enclosure is controlled to be within a predetermined temperature range.

Item 10: In some embodiments according to any of the items 7-9, the second processing enclosure follows the first processing enclosure on the same production line (202).

Item 11: In some embodiments according to any of the items 7-11 wherein the temperature inside the second processing enclosure (102) is measured at a position within a plume or cone of mist generated by the spraying, via the spray nozzle.

Item 12: In some embodiments according to any of the items above, aspects described in the present application are applicable in combination with any of the items above.

It should be noted that the methods mentioned in item 1 and 7 above, may be performed completely independently of each other. It should be noted that the production line or a portion thereof comprising the first and second processing enclosures are generally installed in the one and same building or a collection of semi-detached or detached buildings e.g. a food products factory protecting the production line from the elements. The buildings are located on a coherent piece of real estate.

It should also be noted that the surface temperature of a food product or a surface temperature of a portion of a food product may be measured by a non-contact thermometer sometimes denoted a temperature gun. A non-contact thermometer may give a temperature reading by measuring, at a distance, infrared radiation from one or more points or areas on the food product.

It should be noted that the term ‘food product’ comprises any item that is in an eatable condition, is in a condition for distribution, is in a condition for further processing, or is in a condition of being processed on a production line. As explicated above a ‘food product’ or ‘food products’ may be brought to a production line as livestock, carcasses, harvest, food ingredients, dairy products, etc. Food products are items or objects excluding items or objects in gaseous, liquid or paste form. The food products are in solid form and may have a soft and/or hard surface.

The term ‘surface’ or ‘surface of a food product’ refers to the exterior boundary of the food product comprising an exterior boundary that is curved and faces outwards for convex surfaces or inwards e.g. for concave surfaces.

1. A method for sanitizing food products on a production line, comprising:
conveying food products through a first processing enclosure and onwards through a second processing enclosure;
generating a first processing atmosphere within the first processing enclosure by supplying a flow of gas, with a gas temperature above 70 degrees Celsius, to the first processing enclosure, wherein at least a portion of the surface of a food product is exposed, while travelling through the first processing enclosure, to a first processing temperature (T1) which is above about 60 degrees Celsius;
inside the second processing enclosure, delivering an antimicrobial treatment to the food products when they travel through the second processing enclosure.

2. The method according to claim 1, wherein delivering an antimicrobial treatment to the food products when they travel through the second processing enclosure comprises delivering a spray of an antimicrobial agent towards the food products that travels through the second processing enclosure.

3. The method according to claim 1, wherein the antimicrobial agent is an oxygen-based disinfectant, such as a peroxygen solution.

4. The method according to claim 1, wherein the antimicrobial agent comprises an antimicrobial agent of the biological type, such as salmonella bacteriophages.

5. The method according to claim 1, wherein the antimicrobial agent comprises one or more of: peroxygen compound, sodium hypochlorite, chlorine dioxide, hypochlorous acid, hydrogen peroxide, acetic acid, lactic acid, ozone gas in solution, acidified sodium chlorite, potassium hydroxide, sodium hydroxide, citric acid, and a cationic quaternary ammonium compound, such as cetylpridinium chloride.

6. The method according to claim 1, wherein the surface temperature of the food product is elevated or lowered, while the food product travels through the second processing enclosure, at a rate having a time constant that is relatively short compared to the retention time of the food product in the second processing enclosure.

7. The method according to claim 1, wherein delivering an antimicrobial treatment to the food products when they travel through the second processing enclosure comprises a step of performing rapid surface chilling of the food product.

8. The method according to claim 7, wherein rapid surface chilling is performed by discharging a gas, with a gas temperature below 0° C., inside the second processing enclosure at a sufficient flow rate to cool the surface temperature of at least a portion of the food product to a temperature below about 0° C. within less than about one minute.

9. The method according to claim 1, wherein delivering an antimicrobial treatment to the food products when they travel through the second processing enclosure comprises a step of applying a modified atmosphere wherein the volume-percentage of one or both of Nitrogen and Oxygen deviates from 78.08% and 20.95% by more than 1 percentage points.

10. The method according to claim 1, wherein delivering an antimicrobial treatment to the food products when they travel through the second processing enclosure comprises a step of applying a modified atmosphere packaging, MAP.

11. The method according to claim 1, wherein the food products are exposed to the processing atmosphere in the first processing enclosure for a first processing duration; and wherein the first processing duration is in the range of 0.15 to 15 seconds, 0.15 to 10 seconds, or in the range of 0.2 to 5 seconds, or less than about 4 seconds.

12. The method according to claim 1, wherein the first processing temperature (T1) in the first processing enclosure is in the range of 80 to 95 degrees Celsius.

13. The method according to claim 1, wherein the first processing temperature (T1) is selected from the group of:
above 72 degrees Celsius;
above about 80 degrees Celsius; or
above about 90 degrees Celsius.

14. The method according to claim 1, wherein the surface temperature of a portion of the food product is raised, by the first processing atmosphere, to transiently reach a temperature at or above 72° C. before exiting the first processing enclosure.

15. The method according to claim 1, wherein while travelling through at least a section of the first processing enclosure, a surface temperature of the food product is raised, by an increased surface temperature, (ΔTsf) to transiently reach a temperature at or above 72° C., while a core temperature of the food product raises less than 30%, such as less than 20% or less than 10%, of the increased surface temperature.

16. The method according to claim 1, wherein a surface temperature of a food product is elevated from a first surface temperature measured at a point in time when the food product enters the first processing enclosure to a second surface temperature measured at a point in time when the food product leaves the first processing enclosure; and wherein the food product is conveyed to enter the second processing enclosure before the surface temperature falls below the first surface temperature.

17. The method according to claim 1, wherein the food products are exposed to the first processing atmosphere in the first processing enclosure for a first processing duration, which is sufficiently long to raise the surface temperature of the food product by more than 4 degrees Celsius or more than 10 degrees Celsius.

18. The method according to claim 1, wherein the food products are exposed to the first processing atmosphere in the first processing enclosure for a first processing duration, which is shorter than that required for blanching of the food products at the temperature in the first processing enclosure.

19. The method according to claim 1, wherein the flow of gas comprises steam supplied to the first processing enclosure at a temperature in the range of about 100 to 140 degrees Celsius or in the range of about 120 to 180 degrees Celsius.

20. The method according to claim 1, comprising the step of:
applying airborne high intensity and high power acoustic waves to at least a portion of said first processing atmosphere causing it to oscillate substantially at the frequency and substantially with the intensity and power of the acoustic waves.

21. The method according to claim 20, wherein said high intensity and high power acoustic waves are ultrasonic acoustic waves.

22. The method according to claim 20, wherein said high intensity and high power acoustic waves are generated by a high intensity and high power acoustic wave generator and has an acoustic sound pressure level at approximately 10 cm from an orifice of said generator selected from the group of:
at least 120 dB,
at least 130 dB,
at least 135 dB,
at least 140 dB,
at least 150 dB,
approximately 130 to approximately 165 dB, and
approximately 130 to approximately 180 dB.

23. The method according to claim 1, wherein high intensity sound or ultrasound is generated by a sound generator of the Hartmann type generator and wherein the pressurized gas is supplied to the sound generator at a pressure in the range of 1.5-5 atm.

24. The method according to claim 1, the food products being sanitized are selected from one of the following groups: poultry, meat, warm seafood, cold seafood, warm seafood, vegetables, fruit, lettuce, berries, nuts, cereal, and cheese.

25. A production line for processing food products, comprising:
a first processing enclosure and a second processing enclosure;
a conveyor system configured to move a food product through the first processing enclosure and onwards through the second processing enclosure;
wherein the first processing enclosure is coupled to a gas supply system delivering a flow of gas at a gas temperature above 70 degrees Celsius via an orifice to generate a first processing atmosphere within the first processing enclosure exposing at least a portion of the surface of the food products, while travelling through the first processing enclosure, to a first processing temperature (Ts) which is above 60 degrees Celsius;
wherein the second processing enclosure is configured to deliver an antimicrobial treatment to the food products when they travel through the second processing enclosure.

26. The production line according to claim 25, wherein the second processing enclosure is configured with an atomizing nozzle to deliver a spray of a supply of an antimicrobial chemical agent towards the food products travelling through the second processing enclosure.

27. The production line according to claim 26, wherein the antimicrobial agent is an oxygen-based disinfectant, such as a peroxygen solution.

28. The production line according to claim 25, wherein the second processing enclosure is configured to perform rapid surface chilling of the food product.

29. The production line according to claim 28, wherein rapid surface chilling is performed by discharging a gas, with a gas temperature below 0° C., inside the second processing enclosure at a sufficient flow rate to cool the surface temperature of at least a portion of the food product to a temperature below about 0° C. within less than about one minute.

30. The production line according to claim 25, wherein the second processing enclosure is configured to apply a modified atmosphere wherein the volume-percentage of one or both of Nitrogen and Oxygen deviates from 78.08% and 20.95% by more than 1 percentage points.

31. The production line according to claim 25, wherein the second processing enclosure is configured to apply a modified atmosphere packaging, MAP.

32. The production line according to claim 25, configured to expose the food products to the processing atmosphere in the first processing enclosure for a first processing duration; and wherein the first processing duration is in the range of 0.15 to 10 seconds, or in the range of 0.2 to 5 seconds, or less than about 4 seconds.

33. The production line according to claim 25, wherein the first processing temperature (T1) in the first processing enclosure is in the range of 80 to 95 degrees Celsius.

34. The production line according to claim 25, wherein the flow of gas comprises steam supplied to the first processing enclosure at a temperature in the range of about 100 to 140 degrees Celsius or in the range of about 120 to 180 degrees Celsius.

35. The production line according to claim 25, wherein the first processing enclosure is configured to apply airborne high intensity and high power acoustic waves to at least a portion of said first processing atmosphere causing it to oscillate substantially at the frequency and substantially with the intensity and power of the acoustic waves.

36. The production line according to claim 35, wherein said high intensity and high power acoustic waves are ultrasonic acoustic waves.

37. The production line according to claim 35, wherein said high intensity and high power acoustic waves are generated by a high intensity and high power acoustic wave generator and has an acoustic sound pressure level at approximately 10 cm from an orifice of said generator selected from the group of:
at least 120 dB,
at least 140 dB,
at least 150 dB,
approximately 130 to approximately 165 dB, and
approximately 130 to approximately 180 dB.

38. The production line according to 35, wherein high intensity sound or ultrasound is generated by a sound generator of the Hartmann type generator and wherein the pressurized gas is supplied to the sound generator at a pressure in the range of 1.5-5 atm.

39. The production line according to claim 25, comprising a first storage tank for storing the antimicrobial agent, a second storage tank containing an antimicrobial agent solution, and a compressor for pressurizing the second storage tank and driving the antimicrobial agent solution towards one or more of the nozzles.

40. The production line according to claim 25, wherein the atomizing nozzle is configured to deliver an air assisted induction charged electrostatic spray (AAIC-ES).

41. The production line according to claim 25, comprising a steam generator delivering pressurized steam to the sound generator.

42. The production line according to claim 25, comprising a wall separating a first processing volume enclosed by the first processing enclosure and a second processing volume enclosed by the second processing enclosure wherein the wall has an opening forming a passage through which the conveyor and a food product conveyed thereon can pass.

43. (canceled)
